SEQUENCE LISTING (10 sequences)

SEQ ID NO: 1 - protein, 16 aa, Canis familiaris
  misc_feature (8)..(8): Xaa can be any naturally occurring amino acid
MTKLEDHXEGIVDVFH

SEQ ID NO: 2 - protein, 16 aa, Felis catus
MTKLEEHLEGIINVFH

SEQ ID NO: 3 - protein, 16 aa, Equus caballus
MTKLEDHLEGVINIFH

SEQ ID NO: 4 - protein, 16 aa, Bos taurus
MTKLEDHLEGIINIFH

SEQ ID NO: 5 - protein, 16 aa, Sus scrofa
MTKLEDHLEGIINIFH

SEQ ID NO: 6 - protein, 16 aa, Homo sapiens
MTKLEEHLEGIVNIFH

SEQ ID NO: 7 - protein, 15 aa, Canis familiaris
  misc_feature (7)..(7): Xaa can be any naturally occurring amino acid
TKLEDHXEGIVDVFH

SEQ ID NO: 8 - DNA, 288 bp, Canis familiaris
ctgaagggtgagaagatgactaagctggaggaccacctggaggggatcgttgatgtcttccaccggtactcagcccgggtggggcatcctgacaccctttcaaagggtgagatgaagcagctgatcataagggaacttccaaacaccctcaagaacaccaaagatcaagctacggttgacaaattatttcaagatctggatgctgataaagacggacaggtcaacttcaatgaattcatatccctggtttctgttgtgctagacacttctcataagaatacccacaaa

SEQ ID NO: 9 - protein, 91 aa, Canis familiaris
MTKLEDHLEGIVDVFHRYSARVGHPDTLSKGEMKQLIIRELPNTLKNTKDQATVDKLFQDLDADKDGQVNFNEFISLVSVVLDTSHKNTHK

SEQ ID NO: 10 - protein, 90 aa, Canis familiaris
TKLEDHLEGIVDVFHRYSARVGHPDTLSKGEMKQLIIRELPNTLKNTKDQATVDKLFQDLDADKDGQVNFNEFISLVSVVLDTSHKNTHK

(19) United States
(12) Patent Application Publication
(10) Pub. No.: US 2011/0287458 A1
(43) Pub. Date: Nov. 24, 2011

(54) METHOD AND COMPOSITION FOR THE DIAGNOSIS AND MONITORING OF INFLAMMATORY DISEASES

(75) Inventors: Romy M. Heilmann, College Station, TX (US); Jan S. Suchodolski, College Station, TX (US); Jörg M. Steiner, College Station, TX (US)

(73) Assignee: The Texas A&M University System

(21) Appl. No.: 13/109,760

(22) Filed: May 17, 2011

Related U.S. Application Data
(60) Provisional application No. 61/345,777, filed on May 18, 2010.

(51) Int. Cl. G01N 33/53; C12N 5/10; C12N 15/63; C07K 16/18; C07H 21/04

(52) U.S. Cl. 435/7.94; 530/389.1; 530/388.1; 536/23.5; 435/320.1; 435/325; 436/501; 435/7.9; 435/7.92

(57) ABSTRACT

The present invention provides a canine S100 calcium binding protein useful in diagnosing an inflammatory disease. Also provided are methods of diagnosing an inflammatory disease by assaying a biological sample for the canine S100 calcium binding protein. The invention further provides antibodies specifically binding to the canine S100 calcium binding protein and related kits comprising the antibodies.

This application claims the priority of U.S. Provisional Application No. 61/345,777, filed on May 18, 2010, the entire disclosure of which is incorporated herein by reference.

### BACKGROUND OF THE INVENTION

I. Field of the Invention

The present invention relates in general to the field of diagnosing and monitoring inflammatory diseases by measuring the calcium binding protein expressed predominantly by neutrophils and monocytes, which represents a ligand for the receptor for advanced glycation end products (RAGE) that plays a central role in inflammation.

II. Related Art

There exist a number of diseases that are characterized by symptoms of inflammation (inflammatory diseases). These diseases can affect humans and non-human animals, such as canines. One indication of such inflammatory diseases is the presence of inflammatory cells such as neutrophils and macrophages at local sites of inflammation. The inflammatory state can also be systemic, i.e. proteins secreted by inflammatory cells become detectable in the blood serum.

Laboratory markers represent an alternative indication and may be useful for diagnosis of inflammatory diseases. Such markers can provide an objective measure for the assessment of various diseases. They are non- or minimally invasive and usually simple to perform. Only a few immunological and inflammatory markers have been validated for use in canine patients. There is therefore a lack of markers that are both sensitive and specific for canine patients with chronic inflammatory diseases.

### SUMMARY OF THE INVENTION

In one aspect, the invention provides a method of diagnosing an inflammatory disease in an animal comprising assaying a S100 calcium binding protein level in a biological sample from the animal wherein an increased S100 calcium binding protein level is indicative of an inflammatory disease. Such an inflammatory disease may, in one embodiment, be selected from the group consisting of a systemic disease, a gastrointestinal disease, a urogenital disease, a disease of the central nervous system, inflammatory bowel disease, acute gastroenteritis, chronic gastroenteropathy, parvovirosis, urinary tract infection, inflammatory disease of the central nervous system, and sepsis.

In another embodiment, the biological sample is any biological sample that can be obtained from an animal, for instance, plasma/serum, cerebrospinal fluid, synovial fluid sample, urine, feces or cell culture supernatant. In a further embodiment, the S100 calcium binding protein is selected from S100A8, S100A9, and S100A12. In a particular embodiment the S100 calcium binding protein is canine S100A12. In another embodiment, the S100 calcium binding protein comprises 85% identity to SEQ ID NO:9 or SEQ ID NO:10.

In one embodiment, the methods of the present invention can be performed on a biological sample from any animal. In certain embodiments, the animal may be a canine or porcine. In another embodiment the S100 calcium binding protein level in the biological sample is determined by a competitive immunoassay, a western blot, a radioimmunoassay (RIA), an ELISA (enzyme linked immunosorbent assay), a “sandwich” immunoassay, an immunoprecipitation assay, a precipitin reaction, a gel diffusion precipitin reaction, an immunodiffusion assay, an agglutination assay, an immunoradiometric assay, a fluorescent immunoassay, a protein A immunoassay, an immunoprecipitation assay, an immunohistochemical assay, a competition or sandwich ELISA, a Western blot assay, an immunohistological assay, an immunocytochemical assay, a dot blot assay, a fluorescence polarization assay, a scintillation proximity assay, a homogeneous time resolved fluorescence assay, a IAsys analysis, a BIAcore analysis, or a multiplex immunoassay.

Another aspect of the present invention is an antibody that binds specifically to a canine S100A12 calcium binding protein or an immunogenic portion thereof, for example, a monoclonal or polyclonal antibody. Another aspect of the invention is a nucleic acid molecule encoding a polypeptide with at least 85% identity to a canine S100 calcium binding protein, and in one embodiment the canine S100 calcium binding protein comprises SEQ ID NO:9 or SEQ ID NO:10. In one embodiment, the canine S100 calcium binding protein is selected from S100A8, S100A9, and S100A12, isolated and purified. In another embodiment, the nucleic acid molecule encoding a canine S100 calcium binding protein is operably linked to a heterologous promoter and may be comprised in an expression vector. In a further embodiment, the expression vector may be comprised in a cell such as a cell transfected with the expression vector.

Yet another aspect of the invention is a kit for diagnosing gastrointestinal inflammation in a canine comprising: a) an antibody that specifically binds to at least a fragment of a canine S100 calcium binding protein to form a first binding complex; and b) a detectable label. In one embodiment, the detectable label detects of the presence or absence of the binding complex. In another embodiment, the binding component comprises a monoclonal antibody or a polyclonal antibody. In yet embodiment, the detectable label comprises a radioactive, enzymatic, or colorimetric label. In still another embodiment, the canine S100 calcium binding protein comprises at least one of a S100A8 protein, a S100A9 protein, or a S100A12 protein. In a further embodiment, the kit is a competitive immunoassay, a western blot, a radioimmunoassay, an ELISA (enzyme linked immunosorbent assay), a “sandwich” immunoassay, an immunoprecipitation assay, a precipitin reaction, a gel diffusion precipitin reaction, an immunodiffusion assay, an agglutination assay, an immunoradiometric assay, a fluorescent immunoassay, a protein A immunoassay, an immunoprecipitation assay, an immunohistochemical assay, a competition or sandwich ELISA, a radioimmunoassay, a Western blot assay, an immunohistological assay, an immunocytochemical assay, a dot blot assay, a fluorescence polarization assay, a scintillation proximity assay, a homogeneous time resolved fluorescence assay, a IAsys analysis, a BIAcore analysis, or a multiplex immunoassay.

### BRIEF DESCRIPTION OF THE DRAWINGS

For a more complete understanding of the features and advantages of the present invention, reference is now made to the detailed description of the invention along with the accompanying figures and in which:

FIG. 1A. Graph of the hydrophobic interaction chromatography of the extracted cytosol fraction of canine leukocytes.

FIG. 1B. Graph of the automated fluorescence-based reducing gel electrophoresis (protein 80 assay) of the fractions eluted from the hydrophobic interaction chromatography column.

FIG. 2A. Graph of the cation-exchange column chromatography of fractions containing canine S100A12 from hydrophobic interaction chromatography.

FIG. 2B. Graph of the automated fluorescence-based reducing gel electrophoresis (protein 80 assay) of the fractions eluted from the cation-exchange column.

FIG. 3A. Graph of the anion-exchange column chromatography of canine S100A12 containing fractions following cation-exchange chromatography.

FIG. 3B. Graph of the automated fluorescence-based reducing gel electrophoresis (Protein 80 assay) of purified fractions obtained from anion-exchange chromatography.

FIG. 4. Graph of the SELDI-TOF-mass spectrum of the purified canine S100A12.

FIG. 5A-51. Graphs demonstrating some of the uses of measuring canine S100A12 (calgranulin C, EN-RAGE, MRP6, p6) in samples from dogs.

FIG. 6. Representative calibration plot for the estimation of canine S100A12 (•) by a competitive, double antibody RIA. The gray shaded portion of the curve was used to quantify canine S100A12 in serum and fecal extracts. No cross-reactivity was observed with the canine S100A8/A9 (canine calprotectin) protein complex (∘).

FIG. 7. Scatter plot of serum canine S100A12 concentrations measured in specimens from 124 healthy dogs. Each symbol represents the concentration for a specific dog. Median canine S100A12 concentration (solid horizontal line) and reference interval (gray shaded portion between dashed horizontal lines) were calculated.

FIG. 8. Scatter plot showing the three-day fecal mean canine S100A12 concentrations in specimens collected from 65 healthy dogs. Each symbol represents the three-day mean fecal canine S100A12 concentration for a specific dog. Median (solid horizontal line) and reference interval (gray shaded portion between dashed horizontal lines) for 3-day mean fecal canine S100A12 concentrations were calculated.

FIG. 9. Long-term biological variation of serum canine S100A12 concentrations in 11 healthy dogs. For each dog, the mean (circles) and range (horizontal bars) of serum canine S100A12 concentrations over 2.6 months is shown. The gray shaded area (delineated by dashed vertical lines) indicates the reference interval; the overall mean serum canine S100A12 concentration (77.4 μg/L) is shown by the dotted vertical line. None of the measurements were below the lower detection limit of the RIA (11.2 μg/L; solid vertical line) and all dogs (except for one of the two outlying observation; asterisks) measured below the upper limit of the reference interval (225.1 μg/L); three dogs had canine S100A12 concentrations below the lower limit of the reference interval (33.2 μg/L).

FIG. 10. Short-term biological variation of fecal canine S100A12 concentrations in 65 healthy dogs. The reference interval is indicated by the gray shaded area delineated by its upper limit (745 ng canine S100A12/g feces) to the right (dashed vertical line). In 13 of the 65 dogs (20%) fecal canine S100A12 was detectable in at least one of the samples collected on 3 consecutive days; the majority of the dogs had a three-day sample mean fecal canine S100A12 concentration of <320 ng/g.

FIG. 11. Representative standard curve for the measurement of canine S100A12 by a newly developed sandwich ELISA, calculated using a 5-parameter logistic (5PL) curve fit.

FIG. 12. Biological variation of fecal canine S100A12 concentrations in specimens collected from 53 healthy pet dogs. For each dog, the mean (•) and range for canine S100A12 of fecal samples collected on three consecutive days are shown. The reference interval is indicated by the gray shaded portion between its lower and upper limit (black dashed lines). The red dashed line denotes the lower detection limit of the assay.

FIG. 13. Scatter plot showing the three-day fecal mean canine S100A12 concentrations in specimens collected from 53 healthy pet dogs. Each symbol represents the three-day mean fecal canine S100A12 concentration for a specific dog. Median (solid horizontal line) and control interval (gray shaded portion between dashed horizontal lines) for 3-day mean fecal canine S100A12 concentrations are indicated.

FIG. 14. Correlation of paired fecal canine S100A12 concentrations in samples measured by RIA and ELISA. Concentrations of canine S100A12 measured in 62 fecal samples by both the new ELISA and the previously developed RIA were correlated (Spearman ρ=0.9309), but both a constant and proportional bias existed with lower concentrations for the canine S100A12-RIA compared to the new canine S100A12-ELISA. Each symbol represents the paired canine S100A12 concentrations for a specific fecal sample. The slope of the fitted regression line (solid straight line) was 0.4648 (95% CI: 0.4338-0.4958).

FIG. 15. Bland-Altman plot showing the comparison of fecal canine S100A12 concentrations measured by RIA and ELISA. Each symbol represents the difference between both methods against their mean for a specific fecal sample (n=62). The mean difference (bias) and the lower (LLA) and upper (ULA) limit of agreement (dashed horizontal lines) were calculated as −0.360, −0.980, and 0.260 on the log scale.

FIG. 16. Serum canine S100A12 concentrations in 22 dogs with sepsis measured over a 3-day period during which patients were hospitalized.

FIG. 17A. Serum canine S100A12 concentrations in dogs with chronic enteropathy.

FIG. 17B. Serum canine S100A12 concentrations in dogs with inflammatory bowel disease (IBD) or protein-losing enteropathy (PLE) or food-responsive disease (FRD).

FIG. 18. Fecal canine S100A12 concentrations in puppies with parvovirosis and healthy controls.

FIG. 19A. Urine canine S100A12 concentrations normalized by urine specific gravity (USG) in dogs with urinary tract infections (UTI) and healthy controls.

FIG. 19B. Urine canine S100A12 concentrations normalized by urine creatinine concentration (Cr) in dogs with urinary tract infections (UTI) and healthy controls.

FIG. 20A. Serum canine S100A12 concentrations in dogs with IBD (chronic enteropathy) compared to healthy controls.

FIG. 20B. Fecal canine S100A12 concentrations in dogs with IBD (chronic enteropathy) compared to healthy controls.

FIG. 21. Concentrations of canine S100A12 in cerebrospinal fluid (CSF) from dogs.

### BRIEF DESCRIPTION OF THE SEQUENCE LISTING

SEQ ID NO:1 The first 15 N-terminal residues of the Canis familiaris S100A12 protein.

SEQ ID NO:2 The first 15 N-terminal residues of the Felis catus S100A12 protein.

SEQ ID NO:3 The first 15 N-terminal residues of the Equus caballus S100A12 protein.

SEQ ID NO:4 The first 15 N-terminal residues of the Bos taurus S100A12 protein.

SEQ ID NO:5 The first 15 N-terminal residues of the Sus scrofa S100A12 protein.

SEQ ID NO:6 The first 15 N-terminal residues of the Homo sapiens S100A12 protein.

SEQ ID NO:7 The first 15 N-terminal residues of the purified Canis familiaris S100A12 protein.

SEQ ID NO:8 The full length Canis familiaris S100A12 nucleotide sequence.

SEQ ID NO:9 The full length Canis familiaris S100A12 protein sequence.

SEQ ID NO:10 The full length purified Canis familiaris S100A12 protein.

### DETAILED DESCRIPTION OF THE INVENTION

The present invention provides a canine S100 calcium binding protein, and in one embodiment provides the canine S100A12 protein, comprising SEQ ID NO:1 (the first 15 N-terminal amino acids), SEQ ID NO:7 (the first 15 N-terminal amino acids of the purified protein), SEQ ID NO:9 (the full length canine S100A12 protein sequence) or SEQ ID NO:10 (the full length of the purified canine S100A12 protein). The present invention also provides an immunologic method to assay and quantify canine S100A12 or peptides of this protein in biological samples, such as blood, serum/plasma, cerebrospinal fluid, synovial fluid, urine and feces in an animal, such as a dog (canine). In one embodiment, such a method can be used for diagnosing an inflammatory disease in dogs. As used herein “diagnosing” may refer to the identification or monitoring of a disease. In certain embodiments, diagnosing may refer to the initial indication of a disease and in other embodiments it may refer to further characterization of or monitoring the progression of an already identified disease. An “inflammatory disease” as used herein refers to any disease that is associated with inflammation. In particular embodiments the inflammatory disease may be a disease of the gastrointestinal tract, urogenital tract, respiratory tract, central nervous system or a systemic disease, for instance, comprising inflammatory bowel disease (IBD), chronic enteropathies, gastroenteritis, pancreatitis, hepatitis, cholangitis, rheumatoid arthritis, sepsis, protein-losing enteropathy (PLE), food-responsive disease (FRD), parvovirosis, urinary tract infections (UTI), asthma, autoimmune diseases, glomerulonephritis, meningitis, peritonitis, pleuritis, and vasculitis

The present application therefore provides an analytically validated RIA and ELISA for the assay or quantification of canine S100A12 in canine serum and fecal extracts as a potential clinical marker in dogs with inflammatory conditions, and to evaluate the biological variability of serum canine S100A12 in healthy dogs to determine the utility of a population-based reference interval for serum canine S100A12.

S100A12, also referred to as calgranulin C, p6, extracellular newly identified RAGE-binding protein [EN-RAGE], and migration inhibitory factor-related protein [MRP]-6), which is a calcium binding protein of the S100 superfamily of EF-hand proteins, is a calcium binding protein that is predominantly expressed in neutrophils and monocytes and has been proposed to play a central role in both innate and acquired immune responses. S100A12 functions as a phagocyte specific damage associated molecular pattern (DAMP) molecule and interacts with the receptor for advanced glycation end products (RAGE), a pattern recognition receptor with a central role in inflammation.

A high sequence divergence has been reported for S100A12 proteins from different species, thus substantiating the necessity of using species-specific immunologic methods for the detection of S100A12. The present invention provides a rapid and reproducible protocol for the purification of canine S100A12 from canine whole blood, a partial characterization of this protein, and the development of immunological methods for its detection and quantification in serum and fecal specimens from canine patients. The availability of such an immunological method allows diagnoses of inflammatory diseases, such as IBD, in canine patients.

In veterinary medicine, there is a demand for novel, minimally-invasive or non-invasive markers of inflammation since such markers that are both sensitive and specific are currently lacking. The present invention provides a species-specific immunologic method for the assay and optionally the quantification of canine S100A12 protein. The present invention can be used to detect and quantify the S100A12 protein as a measure of inflammatory disease activity in various biological specimens, such as serum, cerebrospinal fluid, and excretions (e.g., urine and fecal specimens) from canine patients. The present invention (a) will allow to further study the role of canine S100A12 in inflammatory diseases in canine patients; and (b) represents a potentially clinically useful tool for (I) diagnosing canine patients with inflammatory diseases of (a) the gastrointestinal tract, (b) the urogenital tract, (c) the central nervous system, (d) inflammatory diseases of other organs, or (e) those that are systemic; (II) determining the inflammatory disease activity in canine patients with such diseases; and (III) monitoring canine patients with various inflammatory diseases.

### I. Peptides and Polypeptides

Some embodiments of the invention set forth herein pertain to isolated and purified peptides and polypeptides that include a S100 calcium binding protein, such as the canine S100A12 protein, one example of which comprises SEQ ID NO:9 or SEQ ID NO:10. A “polypeptide” as used herein refers to a consecutive amino acid segment of any length. In some embodiments of the present methods, the polypeptides employed therein comprise a sequence of consecutive amino acid residues that includes within its sequence an amino acid sequence having greater than 80% sequence identity, for instance, 80%, 85%, 90%, 95%, 96%, 97%, 98%, 99% or 100% sequence identity to SEQ ID NO:1, SEQ ID NO:7, SEQ ID NO:9 or SEQ ID NO:10. In one embodiment, such proteins have S100 calcium binding protein activity. A “peptide” refers to a compound containing two or more amino acids in which the carboxyl group of one amino acid is linked to the amino group of another amino acid. In a particular embodiment, the peptide or polypeptide, which is a S100 calcium binding protein comprises SEQ ID NO:1, SEQ ID NO:7, SEQ ID NO:9 or SEQ ID NO:10. One of ordinary skill in the art would understand how to generate such a (poly)peptide which is a S100 calcium binding protein in view of the disclosure set forth herein and using any of a number of experimental methods well-known to those of skill in the art. Thus, for instance, substitutions may be made that do not abolish the immunoreactivity of a S100 calcium binding protein.

The term “percent sequence identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (1988); Biocomputing: Informatics and Genome Projects (1993); Computer Analysis of Sequence Data, Part I (1994); Sequence Analysis in Molecular Biology (1987); and Sequence Analysis Primer (1991). Preferred methods to determine identity are designed to give the best match between the sequences tested. Methods to determine identity and similarity are codified in publicly available computer programs. Sequence alignments and percent identity calculations may be performed using, for instance, the Megalign program of the LASERGENE bioinformatics computing suite (DNASTAR Inc., Madison, Wis.), or the like. Multiple alignment of the sequences may be performed as is known in the art, for instance by using the Clustal method of alignment (Higgins and Sharp (1989) with the default parameters (GAP PENALTY=10, GAP LENGTH PENALTY=10). Default parameters for pairwise alignments using the Clustal method are KTUPLE 1, GAP PENALTY=3, WINDOW=5 and DIAGONALS SAVED=5.

It is well understood by the skilled artisan that, inherent in the definition of a “polypeptide,” is the concept that there is a limit to the number of changes that may be made within a defined portion of the molecule and still result in a molecule with an acceptable level of sequence identity, function, e.g., ability to function as a S100 calcium binding protein, or immunologic cross-reactivity.

An amino acid sequence of any length is contemplated within the definition of polypeptide as set forth herein, so long as the polypeptide retains the recited sequence identity. A plurality of distinct S100 calcium binding proteins with different substitutions may easily be made and used in accordance with the invention. Peptidomimetics and peptide analogs that bind to a S100 calcium binding protein are also contemplated. Further, the skilled artisan would know how to design non-peptide structures in three dimensional terms that mimic the peptides that bind to a target molecule.

The present invention may utilize a S100 calcium binding protein purified from a natural source or from recombinantly-produced material. Those of ordinary skill in the art would know how to produce these polypeptides from recombinantly-produced material. This material may use the 20 common amino acids in naturally synthesized proteins, or one or more modified or unusual amino acids. Generally, “purified” will refer to a polypeptide composition that has been subjected to fractionation to remove various other proteins, polypeptides, or peptides, and which composition substantially retains its immunoreactivity. Purification may be substantial, in which the polypeptide is the predominant species, or to homogeneity, which purification level would permit accurate degradative sequencing.

Amino acid sequence variants are encompassed by the present invention, and are included within the definition of “polypeptide.” Amino acid sequence variants of the polypeptide can be substitutional mutants or insertional mutants. Insertional mutants typically involve the addition of material at a non-terminal point in the peptide. This may include the insertion of a few residues; an immunoreactive epitope; or simply a single residue. The added material may be modified, such as by methylation, acetylation, and the like. Alternatively, additional residues may be added to the N-terminal or C-terminal ends of the peptide. Substituted moieties may permit the detection of cleaved peptides by chromogenic, absorbance or fluorescent detection methods.

Amino acid substitutions are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine, and histidine are all positively charged residues; that alanine, glycine, and serine are all a similar size; and that phenylalanine, tryptophan, and tyrosine all have a generally similar shape. Therefore, based upon these considerations, arginine, lysine, and histidine; alanine, glycine, and serine; and phenylalanine, tryptophan, and tyrosine; are defined herein as biologically functional equivalents.

In making changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte and Doolittle, 1982, incorporated by reference herein). It is known that certain amino acids may be substituted for other amino acids having a similar hydropathic index or score and still retain a similar biological activity. In making changes based upon the hydropathic index, the substitution of amino acids whose hydropathic indices are within +2 is preferred, those which are within +1 are particularly preferred, and those within +0.5 are even more particularly preferred.

It is understood that an amino acid can be substituted for another having a similar hydrophilicity value and still obtain a biologically equivalent protein. As detailed in U.S. Pat. No. 4,554,101, the following hydrophilicity values have been assigned to amino acid residues: arginine (+3.0); lysine (+3.0); aspartate (+3.0+1); glutamate (+3.0+1); serine (+0.3); asparagine (+0.2); glutamine (+0.2); glycine (0); threonine (−0.4); proline (−0.5+1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within +2 is preferred, those which are within +1 are particularly preferred, and those within +0.5 are even more particularly preferred.

Changes may also be made based on known substrate specificities for proteases, including aspartic proteases such as pepsin. Combinatorial peptide or polypeptide libraries may also be screened to identify S100 calcium binding proteins, and to assess the relative activity of a S100 calcium binding proteins.

### II. Antibodies and Antibody Fragments

Particular embodiments of the present invention involve antibodies or antibody fragments. The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

“Mini-antibodies” or “minibodies” are also contemplated for use with the present invention. Minibodies are sFv polypeptide chains, which include oligomerization domains at their C-termini, separated from the sFv by a hinge region. Pack et al. (1992). The oligomerization domain comprises self-associating α-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein. Generally, minibodies are produced using recombinant methods well known in the art. See, e.g., Pack et al. (1992); Cumber et al. (1992).

Antibody-like binding peptidomimetics are also contemplated in the present invention. Liu et al., 2003, describe “antibody like binding peptidomimetics” (ABiPs), which are peptides that act as pared-down antibodies and have certain advantages of longer serum half-life as well as less cumbersome synthesis methods.

Monoclonal antibodies (MAbs) are recognized to have certain advantages, e.g., reproducibility and large-scale production, and their use is generally preferred. The invention thus provides monoclonal antibodies of the human, canine, feline, murine, monkey, rat, hamster, rabbit and even chicken origin. Due to the ease of preparation and ready availability of reagents, murine monoclonal antibodies will often be preferred.

However, “humanized” and “caninized” antibodies are also contemplated, as are chimeric antibodies from mouse, rat, or other species, bearing human or canine constant and/or variable region domains, bispecific antibodies, recombinant and engineered antibodies and fragments thereof. As used herein, the terms “humanized” and “caninized” immunoglobulin refer to an immunoglobulin comprising a human or canine framework region, respectively, and one or more complementarity determining regions (CDRs) from a non-human or non-canine (usually a mouse or rat) immunoglobulin. The non-human or non-canine immunoglobulin providing the CDRs is called the “donor” and the human or canine immunoglobulin providing the framework is called the “acceptor”. A “humanized antibody” or “caninized antibody” is an antibody comprising a humanized or caninized light chain and a humanized or caninized heavy chain immunoglobulin, respectively.

The term “antibody” includes polyclonal antibodies (pAbs), monoclonal antibodies (mAbs), chimeric antibodies, anti-idiotypic (anti-Id) antibodies to antibodies that can be labeled in soluble or bound form, as well as fragments, regions or derivatives thereof, provided by any known technique, such as, but not limited to, enzymatic cleavage, peptide synthesis or recombinant techniques. The antibodies set forth herein are capable of binding to a S100 calcium binding protein.

“Polyclonal antibodies” are defined herein to refer to heterogeneous populations of antibody molecules derived from the sera of animals immunized with an antigen. These different antibodies may recognize several epitopes on the same antigen. A “monoclonal antibody” contains a substantially homogeneous population of antibodies specific to antigens, which population contains substantially similar epitope binding sites. MAbs may be obtained by methods known to those skilled in the art. See, e.g., Kohler and Milstein, 1975; U.S. Pat. No. 4,376,110; Ausubel et al., 1992); Harlow and Lane 1988; Colligan et al., 1993, the contents of which are each herein specifically incorporated by reference. Such antibodies may be of any immunoglobulin class including IgG, IgM, IgE, IgA, GILD and any subclass thereof. A hybridoma producing a mAb of the present invention may be cultivated in vitro, in situ, or in vivo. Production of high titers of mAbs in vivo or in situ makes this the presently preferred method of production.

“Chimeric antibodies” are molecules different portions of which are derived from different animal species, such as those having variable region derived from a murine mAb and a human or canine immunoglobulin constant region, which are primarily used to reduce immunogenicity in application and to increase yields in production. Chimeric antibodies and methods for their production are known in the art. Exemplary methods of production are described in Cabilly et al., 1984; Boulianne et al., 1984; and Neuberger et al., 1985, each of which are herein incorporated by reference in their entirety.

An “anti-idiotypic antibody” (anti-Id) is an antibody, which recognizes unique determinants generally associated with the antigen-binding site of an antibody. An anti-Id antibody can be prepared by immunizing an animal of the same species and genetic type (e.g., mouse strain) as the source of the mAb with the mAb to which an anti-Id is being prepared. The immunized animal will recognize and respond to the idiotypic determinants of the immunizing antibody by producing an antibody to these idiotypic determinants (the anti-Id antibody). An exemplary method of producing such antibodies is found in U.S. Pat. No. 4,699,880, which is herein entirely incorporated by reference.

Antibodies of the present invention can include at least one heavy, at least one light chain, a heavy chain constant region, a heavy chain variable region, a light chain variable region and/or a light chain constant region, wherein a polyclonal Ab, monoclonal Ab, fragment and/or regions thereof include at least one heavy chain variable region or light chain variable region that binds a portion of a S100 calcium binding protein, such as the canine S100A12 protein.

As used herein, an antibody is said to “specifically bind” or “immunospecifically recognize” a cognate antigen if it reacts at a detectable level with the antigen, but does not react detectably with peptides containing an unrelated sequence, or a sequence of a different heme protein. Thus, for example, an antibody is said to be “immunospecific” or to “specifically bind” a canine S100A12 polypeptide if it reacts at a detectable level with canine S100A12, e.g., with an affinity constant, Ka, of greater than or equal to about 10⁴ M⁻¹, 10⁵ M⁻¹, 10⁶ M⁻¹, 10⁷ M⁻¹, 10⁸ M⁻¹ or 10⁹ M⁻¹. Affinities of binding partners or antibodies can be readily determined using conventional techniques, e.g., those described by Scatchard et al. (Ann. N.Y. Acad. Sci. USA 51:660 (1949)) or by surface plasmon resonance (BIAcore, Biosensor, Piscataway, N.J.). See, e.g., Wolff et al., Cancer Res. 53:2560-2565 (1993).

In particular embodiments, the antibodies of the present invention may compete for binding to S100 calcium binding protein, such as a canine S100A12 protein. In additional embodiments, antibodies of the present invention may bind specifically to a peptide or protein with an N-terminal sequence of a canine S100A12 protein, for instance comprising SEQ ID NO:1 or SEQ ID NO:7.

Certain embodiments of the present invention pertain to methods for diagnosing an inflammatory disease in an animal that involve obtaining a sample from the animal and assaying the level of S100A12 in the sample. The embodiments may further comprise contacting the sample with an antibody or antibody fragment, wherein the antibody or antibody fragment comprises a domain that binds to one or more S100 calcium binding proteins, to immobilize or at least partially isolate the S100 calcium binding protein. Contacting the putatively S100 calcium binding protein-containing sample by the antibody or antibody fragment may occur prior to, concurrently with, or subsequently to, determining the level of the S100 calcium binding protein in the sample, wherein detection of an elevated level of S100 calcium binding protein as compared to a healthy control is indicative of an inflammatory disease. Any method known to those of ordinary skill in the art can be used to identify the binding of a S100 calcium binding protein and an antibody. Examples of references which address methods for defining variable regions of IgGs include Mo et al. (1993) and Leibiger et al. (1999), herein specifically incorporated by reference.

The antibodies of the present invention can be used, for example, for the immunoprecipitation and quantitation of the canine S100A12 protein of the invention as well as for the monitoring of the presence and/or amount of such canine S100A12 protein. For example, surface plasmon resonance as employed in the BIAcore system can be used to increase the efficiency of phage antibodies that bind to an epitope of the protein of the invention. Antibodies, which bind specifically to a wildtype or a variant protein can be used for diagnosing the disorder taught herein.

### III. Detection Methods and Assay Formats

Certain embodiments of the present invention pertain to methods of diagnosing an inflammatory disease in an animal that involves contacting a sample obtained from an animal with a S100 calcium binding protein, such as canine S100A12. The present invention therefore provides for the use of an antibody in detecting such a S100 calcium binding protein. When used for diagnostic purposes, the peptides and peptide mimetics or antibodies directed against those can be labeled with a detectable label and, accordingly, the peptides and peptide mimetics without such a label can serve as intermediates in the preparation of labeled peptides and peptide mimetics. Detectable labels can be molecules, or compounds, which when covalently attached to the peptides and peptide mimetics, permit detection of the peptide and peptide mimetics. Detection may occur in vivo or in vitro. Detectable labels are well known in the art and may include, for instance, radioisotopes, fluorescent labels (e.g. fluorescein), chemiluminescent labels (e.g. luciferin), other chromogenic substrate or products, and the like. The particular detectable label employed is not critical.

Further, various useful immunodetection methods have been described in the scientific literature, such as, e.g., Nakamura et al. (1987). Immunoassays, in their most simple and direct sense, are binding assays. Certain immunoassays are enzyme linked immunosorbent assays (ELISAs) and radioimmunoassays (RIA). Immunohistochemical detection using tissue sections also is particularly useful. However, it will be readily appreciated that detection is not limited to such techniques, and Western blotting, dot blotting, FACS analyses, and the like also may be used in connection with the present invention.

In general, immunobinding methods include obtaining a sample suspected of containing a protein, peptide or antibody, and contacting the sample with an antibody or protein or peptide in accordance with the present invention, as the case may be, under conditions effective to allow the formation of immunocomplexes. Preferred samples, according to the present invention, are fluids, such as urine, blood, plasma or serum, feces, cerebrospinal fluid, synovial fluid, saliva, peritoneal fluid, pleural fluid, or a cell culture supernatant.

In particular embodiments, the antibody is linked to a solid support, such as the inner wall of a tube, well, or dip stick, and the sample suspected of containing the S100 calcium binding protein will be applied to the immobilized antibody. The specific assay methods described in the embodiments below can be performed using any surface or solid support suitable for coating with an antibody. In certain embodiments, the assay methods described below can be performed using a plate comprising a number of wells or a tube, wherein the sample to be tested and optional additional reagents can be added therein, or a solid support such as a dip stick, wherein the stick is added or dipped into the sample to be tested and optional additional reagents.

Antibody-coated tube systems are described in U.S. Pat. No. 3,646,346 and WO 98/16832, each of which is herein specifically incorporated by reference. Presence of S100-antibody complexes can then be detected under specific conditions. Optionally, such immune complexes can be quantified.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any a S100 calcium binding protein present in the sample. After this time, the sample-antibody composition will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, luminescent, fluorescent, biological, and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Methods for the immunological determination of proteins and kits for carrying out the method can be found in U.S. Pat. No. 5,721,105, herein specifically incorporated by reference.

In particular embodiments, the method involves the use of a secondary binding ligand such as a second antibody and/or a biotin/avidin ligand binding arrangement, as is known in the art. The secondary antibody employed in the detection may itself be linked to a detectable label, wherein one would then simply detect this label, thereby allowing the amount of the primary immune complexes in the composition to be determined. Methods for the detection of a biomolecule in a test sample using immunocapture, biotin/avidin amplification, and horseradish peroxidase color production can be found in U.S. Patent App. Pub. No. 2003/508381.

Usually, the primary immune complexes may be detected by means of a second binding ligand that has binding affinity for the S100 calcium binding protein or the S100 calcium binding protein-specific first antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the S100 calcium binding protein or the S100 calcium binding protein-specific first antibody is used to form secondary immune complexes, as described above. The second binding ligand contains an enzyme capable of processing a substrate to a detectable product and, hence, amplifying signal over time. After washing, the secondary immune complexes are contacted with substrate, permitting detection.

In one embodiment of the invention, enzyme-linked immunoassay (ELISA) may be used. See, e.g., Engvall, 1980; Engvall, 1976; Engvall, 1977; Gripenberg et al., 1978; Makler et al., 1981; Sarangadharan et al., 1984. ELISA allows for substances to be passively adsorbed to solid supports such as plastic to enable facile handling under laboratory conditions. For a comprehensive treatise on ELISA the skilled artisan is referred to “ELISA; Theory and Practice” (Crowther, 1995).

The sensitivity of ELISA methods is dependent on the turnover of the enzyme used and the ease of detection of the product of the enzyme reaction. Enhancement of the sensitivity of these assay systems can be achieved by the use of fluorescent and radioactive substrates for the enzymes. Immunoassays encompassed by the present invention include, but are not limited to those described in U.S. Pat. No. 4,367,110 (double monoclonal antibody sandwich assay) and U.S. Pat. No. 4,452,901 (Western blot). Other assays include immunoprecipitation of labeled ligands and immunocytochemistry, both in vitro and in vivo.

In one embodiment, the invention comprises a “sandwich” ELISA, where anti-S100 antibodies are immobilized onto a selected surface, such as a well in a polystyrene microtiter plate, a tube, or a dip stick. Then, a test composition suspected of containing S100 calcium binding proteins, e.g., a biological sample, is contacted with the surface. After binding and washing to remove non-specifically bound immunocomplexes, the bound antigen may be detected by contacting the complex with a second antibody to the S100 calcium binding protein.

In another exemplary ELISA, polypeptides from the sample are immobilized onto a surface and then contacted with an anti-S100 antibody. After binding and washing to remove non-specifically bound immune complexes, the bound antigen-antibody complex is detected by an immunoassay. Where the initial antibodies are linked to a detectable label, the primary immune complexes may be detected directly. Alternatively, the immune complexes may be detected using a second antibody that has binding affinity for the first antibody, with the second antibody being linked to a detectable label.

Another ELISA in which the S100 calcium binding proteins are immobilized involves the use of antibody competition in the detection. In this ELISA, antibodies specific to S100 calcium binding proteins are added to the wells, allowed to bind to the S100 calcium binding proteins, and detected by means of their label. The amount of S100 calcium binding protein in a sample is determined by mixing the sample with the labeled antibodies before or during incubation with coated wells. The presence of S100 calcium binding protein in the sample acts to reduce the amount of antibody available for binding to the well, and thus reduces the ultimate signal.

Irrespective of the format employed, ELISAs have certain features in common, such as coating, incubating or binding, washing to remove non-specifically bound species, and detecting the bound immune complexes. In coating a plate, well, tube, or dip stick with either antigen or antibody, one will generally incubate the coated surface with a solution of the antigen or antibody, either overnight or for a specified period of hours. The surface will then be washed to remove incompletely adsorbed material. Any remaining available surfaces of the surface are then “coated” with a nonspecific protein that is antigenically neutral with regard to the test antisera. These can include bovine serum albumin (BSA), casein, solutions of milk powder or other antigenically neutral proteins. The coating allows for blocking of nonspecific adsorption sites on the immobilizing surface and thus reduces the background caused by nonspecific binding of antisera onto the surface.

“Under conditions effective to allow immune complex (antigen/antibody) formation” means that the conditions preferably include diluting the antigens and antibodies with solutions such as BSA, bovine gamma globulin (BGG), evaporated or powdered milk, and phosphate buffered saline (PBS)/Tween or Tris buffered saline (TBS)/Tween. These added agents also tend to assist in the reduction of nonspecific background.

The “suitable” conditions also mean that the incubation is at a temperature and for a period of time sufficient to allow effective binding. Incubation steps are typically from about 1 h to 2 h to 4 h, at temperatures preferably on the order of 25° C. to 37° C., or may be overnight at about 4° C. or so.

After incubation with the labeled antibody, and subsequent to washing to remove unbound material, the amount of label is quantified, e.g., by incubation with a chromogenic substrate such as urea and bromocresol purple or 2,2′-azido-di-(3-ethyl-benzothiazoline-6-sulfonic acid (ABTS) and H₂O₂, in the case of peroxidase as the enzyme label. Quantitation is then achieved by measuring the degree of color generation, e.g., using a visible spectra spectrophotometer.

### IV. Purification of Proteins

Certain embodiments pertain to an isolated or purified polypeptide, or methods employing an isolated or purified polypeptide. Protein purification techniques are well known to those of skill in the art. These techniques involve, at one level, the crude fractionation of the cellular milieu of polypeptide and non-polypeptide fractions. Having separated the polypeptide of interest from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, size exclusion chromatography, hydrophobic interaction chromatography, polyacrylamide gel electrophoresis, and isoelectric focusing. A particularly efficient method of purifying peptides is fast protein liquid chromatography (FPLC) or even high pressure liquid chromatography (HPLC).

Certain aspects of the present invention concern the purification, and in particular embodiments, the substantial purification, of an encoded protein or polypeptide. The term “purified polypeptide, protein, or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various techniques suitable for use in protein purification will be well known to those of skill in the art. These include, for example, precipitation with ammonium sulfate, PEG, antibodies and the like or by heat or acid pH denaturation of contaminating proteins, followed by centrifugation; chromatography steps such as ion exchange, gel filtration, reverse phase, hydroxylapatite, and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the polypeptide always be provided in its most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the functionality of a protein.

### V. Kits

In still further embodiments, the present invention provides kits for use with the detection methods described above for the detection of S100 calcium binding proteins, for instance, comprising an immunodetection kit, to diagnose an inflammatory disease in an animal, such as a canine. The kit may include one or more containers. The container, for example, may be a vial, a tube, a flask, a vial, or a syringe.

In further embodiments, the kit includes one or more tubes or wells of a microtiter plate with prebound antibody. Alternatively, the kit may include antibody prebound to a column matrix. The kit may allow for the assay of a single sample, or more than one sample. In some embodiments, the kit includes a plurality of microtiter plates or tubes coated with antibody, which allow for immunodetection of numerous samples concurrently or consecutively. In particular embodiments, the antibody is a polyclonal antibody, a monoclonal antibody, or other characterized anti-S100 immunoglobulin.

The immunodetection reagents of the kit may take any one of a variety of forms, including those detectable labels that are associated with and/or linked to the given antibody. Detectable labels that are associated with and/or attached to a secondary binding ligand are also contemplated. Exemplary secondary ligands are those secondary antibodies that have binding affinity for the first antibody.

In some embodiments, the kits include a secondary antibody that has binding affinity for the first antibody. The second antibody may or may not be linked to a detectable label. In some further embodiments, the kit includes a third antibody that has binding affinity for the second antibody, the third antibody being linked to a detectable label. As noted above, a number of exemplary labels are known in the art and/or all such labels may be employed in connection with the present invention.

The kits may optionally include a suitably aliquoted composition of a S100 calcium binding protein to provide for a positive control. The components of the kits may be packaged either in aqueous media and/or in lyophilized form.

### EXAMPLES

The following examples are included to demonstrate preferred embodiments of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments, which are disclosed and still obtain a like or similar result without departing from the spirit and scope of the invention.

### Example 1

### Isolation and Characterization of Canine S100A12

### I. Materials and Methods

Leukocytes were isolated as previously reported and incorporated herein. Briefly, after mixing two volumes of ethylenediamine tetraacetic acid (EDTA) blood with one volume of a 6% (w/v) dextran (T-500) solution containing 0.9% (w/v) NaCl and sedimentation at room temperature (approximately 23° C.) for 180 minutes, the supernatant layer was collected and leukocytes were pelleted by centrifugation at 2,000×g for 10 minutes at 4° C. Remaining erythrocytes were lysed by incubating the sediment with RBC lysis solution (Qiagen, Valencia, Calif., USA), followed by centrifugation as described above and storage of the pellets at −80° C. until further use.

Leukocyte pellets were resuspended in 20 mM Tris-HCl, 50 mM NaCl, pH 7.6 containing 15 mM diisopropylfluorophosphate (DFP) and the cytosol fraction was extracted as has been described previously. Briefly, after homogenizing the solution for 5 minutes at room temperature (approximately 23° C.), the cell suspension was sonicated for 12 minutes at 150 W on ice and subjected to two successive cycles of freezing (−20° C.), thawing and sonication. The soluble cytosolic fraction was then separated from cell debris by centrifugation at 16,000×g for 12 minutes at 4° C.

The soluble cytosol fraction of leukocytes was precipitated by adding ammonium sulfate to a final concentration of 30% (w/v). After centrifugation at 12,000×g for 25 minutes at 1° C., the supernatant was collected and adjusted to a final concentration of 2 mM CaCl₂. Afterwards, the solution was filtered through a 0.1 μm-pore size filter and applied to a phenylsepharose HIC column (HiTrap™ Phenyl FF, GE Healthcare, Piscataway, N.J., USA) equilibrated with 20 column volumes of 20 mM Tris-HCl, 2 mM CaCl₂, 1.55 M (NH₄)₂SO₄, pH 7.6 at a flow rate of 0.5 ml/minutes at room temperature on a fast protein liquid chromatography (FPLC) system (AKTA basic, GE Healthcare). Proteins bound to the column were eluted in a linear gradient of 0-100% 20 mM Tris-HCl, 5 mM ethylene glycol tetraacetic acid (EGTA), pH 7.6 over 200 minutes, and were analyzed by automated fluorescence-based reducing gel electrophoresis in a chip format (Protein 80 assay) using a Bioanalyzer 2100 system according to the manufacturer's instructions (Agilent Technologies, Palo Alto, Calif., USA).

Canine S100A12 containing fractions eluted from the HIC column were pooled and dialyzed against 50 mM CH₃CO₂Na, 1 mM EDTA, 1 mM EGTA, 1 mM dithiothreitol (DTT), 50 mM NaCl, pH 5.0 (buffer A) using dialysis bags (2,000 Mw cutoff; Thermo Scientific, Waltham, Mass., USA), clarified by 0.1 μm-filtration and subjected to strong cation-exchange chromatography. The column (ECONO-PAC® High S, Bio-Rad Laboratories, Hercules, Calif., USA) was equilibrated with 25 column volumes buffer A at a flow rate of 1 ml/minute at approximately 23° C., and fractions were eluted with a linear gradient of 0.05-0.5 M NaCl in buffer A over 120 minutes and were again analyzed by the Protein 80 assay.

Fractions containing canine S100A12 were pooled and dialyzed against 50 mM Tris-HCl, 1 mM EDTA, 1 mM EGTA, 1 mM DTT, 25 mM NaCl, pH 8.5 (buffer B) using dialysis cassettes (2,000 Mw cutoff; Thermo Scientific), filtered (0.1 μm pore size) and applied to a strong anion-exchange chromatography column (HiPrep™-Q, GE Healthcare) equilibrated with 12 column volumes buffer B at a flow rate of 1 ml/minute at room temperature. Elution of proteins bound to the column was achieved by application of a linear gradient of 0.025-0.3 M NaCl in buffer B over 180 minutes, and the fractions eluted were again analyzed by the Protein 80 assay. Fractions containing canine S100A12 were pooled, dialyzed against 50 mM NH₄HCO₃, pH 7.64 (buffer C) and lyophilized (Freeze Dry System, Labconco, Kansas City, Mo., USA), followed by re-dissolution in 20 mM CH₃CO₂Na, 3 mM CaCl₂, pH 7.6 (buffer D) and storage at −80° C.

Protein purity was assessed by reducing sodium dodecyl sulfate polyacrylamide gel electrophoresis (SDS-PAGE, 10% Bis/Tris) in a vertical mini-gel format (Invitrogen, Carlsbad, Calif., USA), and protein bands were visualized by staining with Coomassie brilliant blue R-250 (Thermo Scientific) according to the manufacturers' instructions.

Native polyacrylamide gel electrophoresis (PAGE) was performed by use of the MINI PROTEAN® 3 electrophoresis cell and a discontinuous buffer system (Ornstein-Davis) according to the standard procedures given by the manufacturer (Bio-Rad Laboratories). Using native PAGE, pure canine S100A12 was evaluated in the presence of 0.3 mM Ca²⁺, 3 mM Ca²⁺, 10 mM Ca²⁺, and 10 mM EGTA.

Protein concentration was measured using the Bradford dye-binding method with bovine serum albumin (BSA) as a reference protein in accordance with the manufacturer's instruction (Bradford protein assay, Thermo Scientific).

Following reducing SDS-PAGE, electroblotting of proteins onto 0.2-1 μm pore size polyvinylidene fluoride (PVDF) membranes was performed as described elsewhere. Membranes were then blocked overnight at 4° C. by immersion in 10 mM Tris-HCl, 150 mM NaCl, 0.05% (v/v) polyoxyethylene sorbitan monolaurate, pH 7.6 (blot buffer) supplemented with 5% (w/v) BSA (blocking buffer). All subsequent steps were carried out at approximately 23° C. on an orbital shaker at 80 rpm, unless otherwise stated. Blots were incubated for 3 h with the rabbit polyclonal anti-recombinant human (rh) S100A12 antiserum (Novus Biologicals, Littleton, Colo., USA) at a dilution of 1:300 or with the rabbit polyclonal anti-rh S100A12 ab37657 (Abcam, Cambridge, Mass., USA) at a dilution of 1:250 in blocking buffer, then washed 4×5 minutes in blot buffer, and incubated with biotin-conjugated goat anti-rabbit IgG (Thermo Scientific) as secondary antibody for 2.5 h (dilution of 1:10,000 in blocking buffer). After 4×5 minute washes with blot buffer, the membranes were incubated for 2 h with horseradish peroxidase-conjugated NeutrAvidin™ (Thermo Scientific) at a concentration of 0.3 mg/l in blot buffer supplemented with 5% (w/v) BSA. The membranes were washed in blot buffer, and the antigen-antibody complexes were detected by incubation for 20 minutes at 37° C. with a stabilized 3,3′,5,5′-tetramethylbenzidine blotting substrate (Thermo Scientific).

Automated fluorescence-based reducing gel electrophoresis (Protein 80 assay; Agilent Bioanalyzer 2100) was used to estimate the molecular weight (Mw) of canine S100A12.

The relative molecular mass (Mᵣ) of canine S100A12 was estimated by surface-enhanced laser desorption/ionization time-of-flight mass spectrometry (SELDI-TOF-MS; ProteinChip® SELDI System, Bio-Rad Laboratories). Briefly, 220 ng of purified canine S100A12 were immobilized onto a Normal Phase (NP) 20 ProteinChip® array using 12.5 mg/ml 3,5-dimethoxy-4-hydroxycinnamic acid (sinapinic acid, SPA) in acetonitrile (ACN):H₂O:TFA (50:49.5:0.5) to allow desorption/ionization of the protein. Ten different TOF spectra were generated by 210 laser shots with a laser intensity of 1,400 nanojoules (nJ) each, and Mr accuracy was calibrated externally by use of the All-in-One protein standard solution covering a mass-to-charge ratio (m/z) range between 6,964 and 147,300.

The isoelectric point (pI) was estimated by native isoelectric focusing (IEF) on a vertical format pre-cast polyacrylamide IEF gel covering a linear pH gradient from 3 to 10 (Invitrogen). Hereafter, proteins were applied to the second dimension (2D-PAGE) consisting of native and non-reducing SDS-PAGE (12% Tris-glycine, Invitrogen), respectively. Protein bands were visualized by Coomassie blue stain.

The specific absorbance of canine S100A12 was determined by the quotient of the spectrophotometric absorbance of serially diluted samples containing pure canine S100A12 measured at a wavelength of 280 nm (NANODROP™ 1000, Thermo Scientific) and the corresponding protein concentrations as determined by a Bradford assay.

Surface-enhanced laser desorption/ionization time-of-flight mass spectrometry (SELDI TOF-MS) was used for the fingerprinting of peptides generated by tryptic digestion (PMF). Following SDS-PAGE, Coomassie blue staining and SDS removal, an in-gel proteolytic digestion was performed by incubation of the excised and destained protein band in 20 μg/ml bovine pancreatic trypsin in 22.5 mM ammonium bicarbonate, 1 mM HCl, pH 8.0 for 16 h at 37° C. Then, 2 μl of the extraction mixture were applied to a ProteinChip® NP20 array using 200 mg/ml alpha-cyano-4-hydroxy cinnamic acid (CHCA) in ACN:H₂O:TFA (50:49.5:0.5) as a matrix to facilitate desorption and ionization of peptides. Ten different TOF spectra were acquired at 210 laser shots with a laser intensity of 900 nJ each, and Mr accuracy was calibrated externally by use of the ProteinChip® Peptide mass standard array covering a mass range between 1,084 and 5,964 (Bio-Rad Laboratories). After peptide masses were extracted from the mass spectra by use of the PeakErazor software, in silico analysis was performed by searching the data obtained against a comprehensive database using the ProFound search site. The mass tolerance for matching peptide average masses was set at 100 ppm, and a maximum of 1 missed cleavage of trypsin was allowed in the search that was restricted to the species (Canis familiaris) as well as the biochemical properties (pI and mass range) of the protein.

The N-terminal amino acid (AA) sequence of canine S100A12 was analyzed by the Edman degradation method on a Model 492 automated protein sequencing system (Applied Biosystems, Foster City, Calif., USA) at the Protein Chemistry Laboratory (Department of Biochemistry and Biophysics, Texas A&M University, College Station, Tex., USA), followed by sequence comparison against a comprehensive database.

### II. Results

The summary of an exemplary purification procedure of canine S100A12 is presented in Table 1. An SDS-PAGE was performed using samples collected after each purification step and revealed a single band in the expected Mw range for canine S100A12 (7,300) for the samples at each step.

TABLE 1
Purification of canine S100A12. This table shows the sequential
purification of canine S100A12 from 900 ml of canine whole blood
(data representative of three separate purification procedures).
Purification of canine S100A12 after various purification steps

| | Total protein |
|---|---|
| Purification stage | content (mg) |
| Extraction of leukocyte cytosol fraction | 165.3 |
| Ammonium sulfate precipitation | 119.2 |
| Hydrophobic interaction chromatography | 7.7 |
| Cation-exchange column chromatography | 1.9 |
| Anion-exchange column chromatography | 1.5 |
| Concentration (lyophilization and storage) | 1.5 |

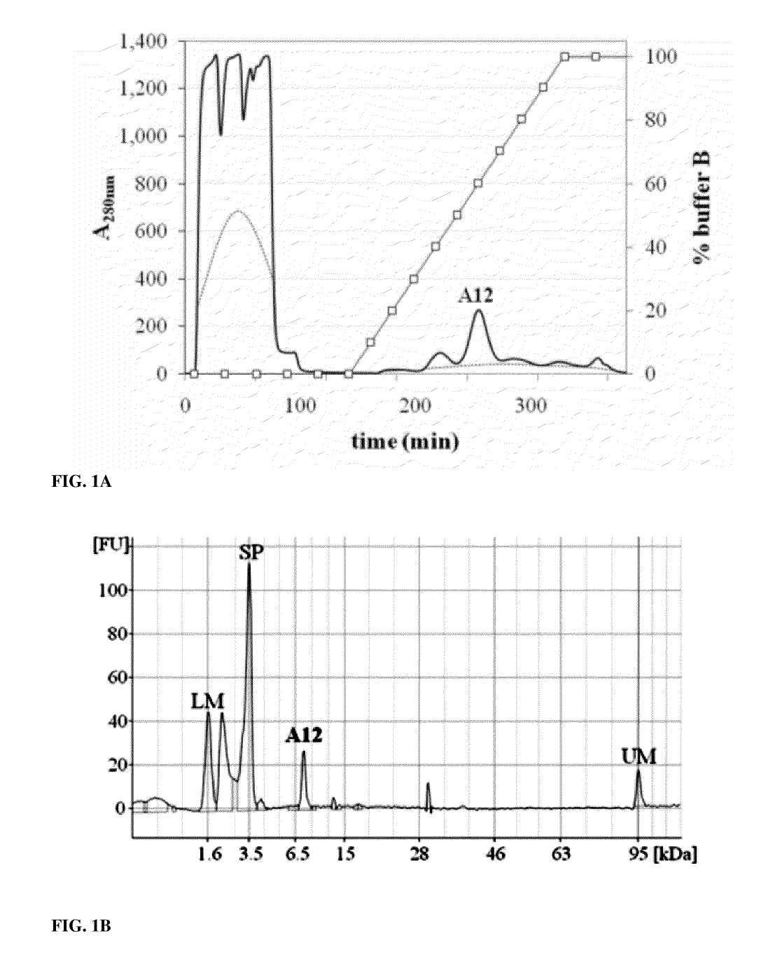

The extracted cytosol fraction of canine leukocytes containing a maximum of 200 mg total protein was subjected to ammonium sulfate precipitation followed by injection onto a phenylsepharose hydrophobic interaction chromatography cartridge, where the majority of canine S100A12 was eluted at approximately 53.3% buffer B (FIG. 1A).

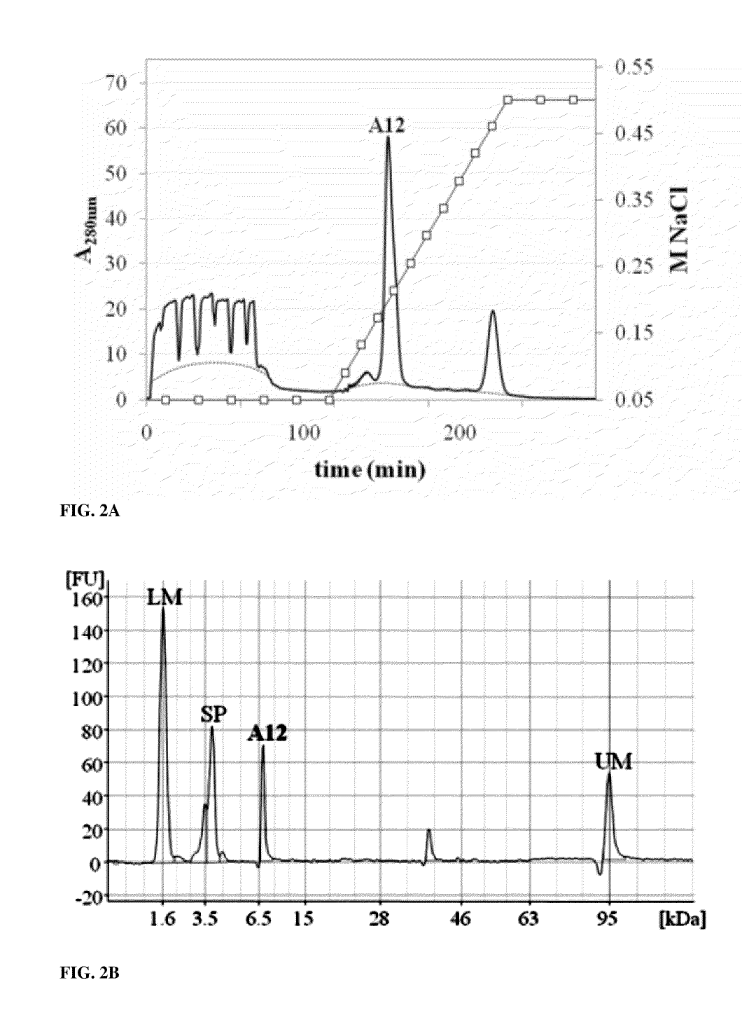

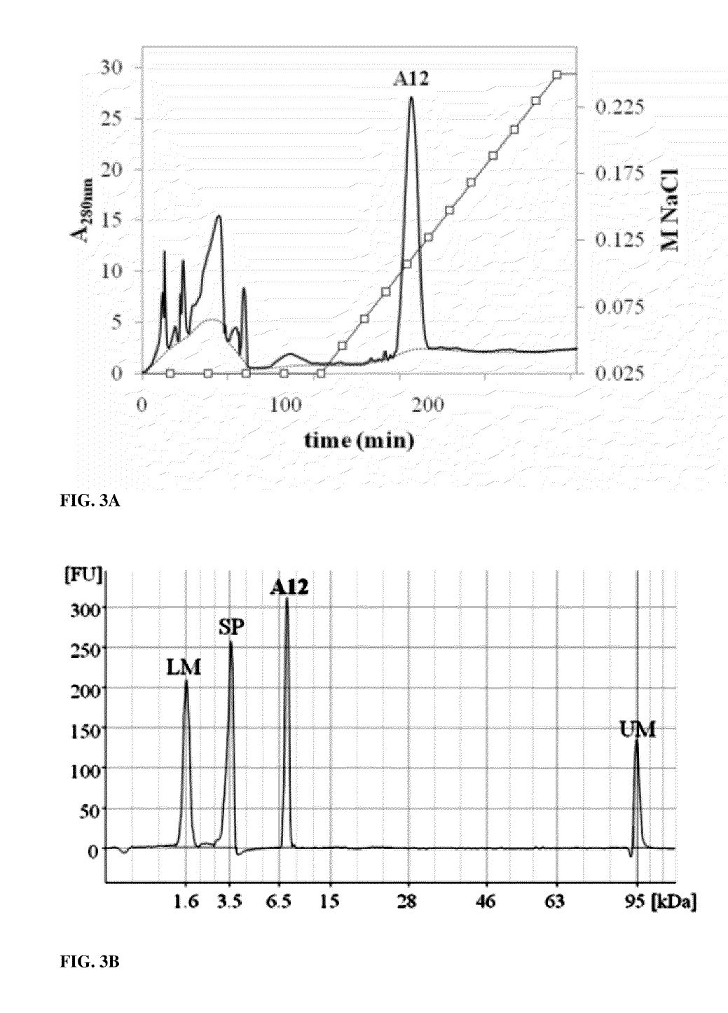

FIG. 1A is an image of hydrophobic interaction chromatography of the extracted cytosol fraction of canine leukocytes. Proteins bound to the column were eluted by a linear gradient of 1.55 to 0.0 M (NH₄)₂SO₄, 2.0 to 0.0 M CaCl₂, and 0.0 to 5.0 mM EGTA in buffer A (open squares). The protein elution profile was obtained by UV absorption at 280 nm (bold line). Peak labeled A12: canine S100A12. FIG. 1B is an image of Protein 80 assay (reducing conditions) of the fractions eluted from the hydrophobic interaction chromatography column. The majority of canine S100A12 eluted in a single major peak (labeled A12). Migration time (tM) proportional to the size (Mw) of the proteins is indicated on the x-axis. A standard curve of tM versus Mw is plotted and used to calculate Mw for each fragment in the sample based on tM measured. Mw markers: 1,600 (lower marker, LM), 3,500 (system peak, SP; quality control for each sample run), 6,500, 15,000, 28,000, 46,000, 63,000, and 95,000 (upper marker, UM). Fractions containing canine S100A12 were further fractionated on an S-SEPHAROSE™ cation exchange and a Q-SEPHAROSE™ anion-exchange column, where canine S100A12 eluted in a symmetric peak each at approximately 0.19 M NaCl and 0.09 M NaCl, respectively (FIG. 2A and FIG. 3A).

FIG. 2A shows cation-exchange column chromatography of fractions containing canine S100A12 from hydrophobic interaction chromatography. Bound proteins were eluted by a linear NaCl gradient of 0.05-0.50 M in buffer A (open squares). The protein elution profile was obtained by UV absorption at 280 nm (bold line). Peak labeled A12: canine S100A12. FIG. 2B illustrates a protein 80 assay (reducing conditions) of the fractions eluted from the cation-exchange column. Canine S100A12 (peak A12) eluted in a single major peak.

FIG. 3A illustrates an anion-exchange column chromatography of canine S100A12-containing fractions following cation-exchange chromatography. Proteins bound to the column were eluted with an increasing concentration of buffer B containing 0.3 M NaCl (open squares) and monitored at 280 nm absorbance (bold line). Peak labeled A12: canine S100A12. FIG. 3B illustrates a Protein 80 assay (reducing conditions) of purified fractions obtained from anion-exchange chromatography. Canine S100A12 (peak A12) was localized in one peak.

During the Protein 80 assay, canine S100A12 revealed a distinct peak at approximately 7,730 (FIG. 1B, FIG. 2B, and FIG. 3B). Once polyclonal antibodies (pAb) were produced in rabbits (described in Example 2, below), the progress of the purification procedure could be monitored by Western blot analysis. With the detection system used, the rabbit anti-canine S100A12 antiserum strongly recognized canine S100A12, and there was no cross-reactivity observed with either the complex of canine S100A8/A9 (canine calprotectin) or canine S100A8 alone. Using the pAb anti-rhS100A12 and ab37657, however, did not result in the detection of any protein bands. The overall yield of the purification protocol was 1.5 mg per 900 ml canine whole blood, and similar proportions were obtained by repeating the protocol two times. Stability of purified canine S100A12 in buffer D (20 mM CH₃CO₂Na, 3 mM CaCl₂, pH 7.6) was demonstrated after storage at −80° C. for 9 months by means of the Bradford assay and SDS-PAGE.

Due to the restricted Mw analysis range of the Protein 80 assay (5,000-80,000), purity of canine S100A12 in buffer D (storage buffer) was evaluated by SDS-PAGE (under reducing conditions) and revealed a single band in the expected Mw range for canine S100A12 (7,300). On SDS-PAGE under non-reducing conditions, a single protein band consistent with the respective monomeric form of canine S100A12 was also observed. However, native PAGE revealed a major protein band consistent with the heterodimeric form of canine S100A12 and this was seen regardless of the presence of Ca²⁺ ions or EGTA in the buffer (16,800). A very minor fraction of the protein migrated as a homotetramer (with a Mw of approximately 36,800) on native PAGE when the buffer contained 10 mM Ca²⁺.

Partial characterization of canine S100A12. The Mr of canine S100A12 was estimated at 10,379.5, and a doublet peak corresponding to the monomer of canine S100A12 was the most intense signal in each of the spectra. A minor doublet peak detected was assigned to a non-specific double charged monomer of the protein (FIG. 4), and reducing SDS-PAGE excluded the possibility that this minor peak may have arisen from a contaminating substance.

FIG. 4 shows that the SELDI-TOF-mass spectrum of the purified canine S100A12. Mᵣ (m/z) range was 5,000-40,000. To ensure accurate protein Mᵣ assignments, generated TOF-mass spectra were externally calibrated using Mᵣ standards (recombinant hirudin (6,964), bovine cytochrome c (12,230), equine cardiac myoglobin (16,951), bovine RBC carbonic anhydrase (29,023), yeast (S. cerevisiae) enolase (46,671)). A doublet peak corresponding to the singly charged canine S100A12 monomer (m/z: 10,379.5; peak A12) yielded the most intense signal. A non-specific double charged monomer of the protein was also detected (m/z approximately 5,178.3), but no other multimeric forms were evident.

Isoelectric focusing of canine S100A12 revealed a single band in the presence as well as the absence of Ca²⁺ or EGTA in the solution, which migrated as a homodimer in the second dimension consisting of native PAGE compared to a monomer during SDS-PAGE. The detected band had an approximate pI of 6.0, which migrated to a 6.9 kDa position in the second dimension on a gel containing SDS and to a 15.8 kDa position in the second dimension on a native gel.

The approximate specific absorbance of canine S100A12 at 280 nm was determined to be 1.78 for a 1 mg/ml solution. Characterization of peptides generated by tryptic digestion revealed the separation of 16 different peaks with m/z values of 955, 1,399, 1,615, 1,679, 1,864, 1,909, 2,072, 2,275, 2,528, 2,874, 2,987, 3,217, 3,286, 3,629, 4,252, and 4,861. As determined by in silico comparison, the m/z ion values of these peaks correlated well with the expected m/z values of digest fragments for the complete AA sequence of canine S100A12 and covered 68% of the total length of the AA sequence of the protein.

The AA sequence of the first 15 N-terminal residues was obtained for this protein, with the single letter code SEQ ID NO:7 TKLEDHXEGIVDVFH(X denotes the AA at position 7, which could not be unambiguously identified) (Table 2). A database search using the 14 AA analyzed revealed complete identity with the primary sequence predicted from the copy DNA (cDNA) sequence for canine S100A12 with the exception that the first methionine residue was cleaved off from the protein. Thus, the protein purified was identified as pure canine S100A12. The highest homology was found with canine S100A8 and canine S100A9 (38.5 and 44.6% identity, respectively).

TABLE 2
N-terminal amino acid (AA) sequences of canine, feline, equine, bovine, porcine and human S100A12 (SEQ ID
Nos: 1-6, respectively).

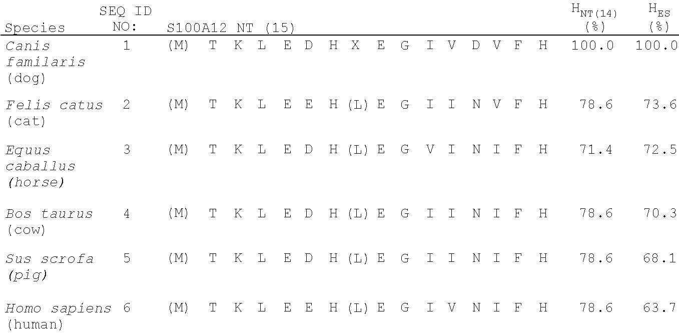

The canine S100A12 protein showed greatest homology with canine S100A8 (MRP8) and S100A9 (MRP14), both of which are consistent with those reported for human S100A12 (40% and 46% identity with human S100A8 and human S100A9, respectively). In this study, pAb raised against canine S100A12 did not detect purified canine S100A8 or the canine S100A8/A9 complex as determined by radial immunodiffusion. While this finding is consistent with that for the respective human counterparts in one study, it differs with those of other studies where polyclonal anti-human S100A12 was found to be slightly cross-reactive to human S100A8 or the human S100A8/A9 complex. Based on this finding, we speculate that the rabbit anti-canine S100A12 pAb produced in this study is of valuable potential for the development of an analytically specific immunologic method for the detection of canine S100A12.

The exposure of hydrophobic domains of the effector protein in order to interact with the respective targets has been shown for human S100A12 and several other proteins belonging to the S100 superfamily. Leading to an altered chromatographic behavior of canine S100A12 in the presence of Ca²⁺, this property was taken advantage of during the initial step of the purification procedure consisting of hydrophobic interaction chromatography, where canine S100A12 was bound in the presence of 2 mM Ca²⁺ and could be eluted from the cartridge by an increasing concentration of a Ca²⁺-free buffer containing 5 mM EGTA. To prevent any artifactual interaction with other reagents or materials due to an increased hydrophobicity in the presence of Ca²⁺, the buffers employed throughout the remaining purification stages contained DTT, EDTA, and EGTA, whereas the amount of Ca²⁺ in the storage buffer (3 mM) was chosen at a concentration similar to that found in extracellular biological fluid.

The pI value observed for canine S100A12 is in agreement with the expected p/calculated based on the AA sequence (5.9) and compares to that reported for human and porcine S100A12. However, unlike in humans and porcines, only one isoform was found for canine S100A12. As the two different isoforms of the human counterpart have been demonstrated to exert different affinities for the binding of Ca²⁺, the possibility that canine S100A12 molecules with a lower Ca²⁺-binding affinity may have been eliminated during the HIC step of the purification procedure in this study cannot be ruled out.

The Mw of canine S100A12 was determined to be approximately 7,730, which is consistent with the M_{M} estimated by SDS-PAGE. As some Ca²⁺-binding proteins have been reported to show aberrant mobilities on SDS-PAGE gels, such as human S100A12 due to its distinct nonspherical shape, a more precise determination of the relative molecular mass (Mᵣ) of canine S100A12 was aimed at using SELDI-TOF-MS analysis. Although purified canine S100A12 appeared to be homogeneous during isoelectric focusing (IEF), Protein 80 assay, native and SDS-PAGE (revealing only one single band or peak formation, respectively) and N-terminal AA sequencing, SELDI-TOF-MS spectra unveiled the presence of two components. The m/z value of 10,379.5 that was revealed for the major component of canine S100A12, is in good agreement with the expected masses derived from the AA sequence in the absence of the first methionine residue (10,376), and compares to the Mr of human S100A12 as determined by SELDI-TOF-MS (10,444) and electrospray ionization mass spectrometry (ESI-MS; 10,444) but also to that of porcine (10,614 and 10,654), bovine (10,554), and rabbit S100A12 (10,680). The absence of the N-terminal methionine residue has also been reported for human, porcine, bovine, and rabbit S100A12.

The presence of a doublet peak with a difference in mass of about 208.2 and an m/z value for the second minor peak of approximately 10,587.7, could be speculated to indicate posttranslational modification of a minor fraction of canine S100A12. This difference being the result of divalent cation binding to the monomer cannot be ruled out but seems rather unlikely, especially because a buffer devoid of any such ions with the exception of Ca²⁺ (m/z≈40) was used. A doublet peak has also been found for porcine S100A12 using electrospray ionization mass spectrometry (ESI-MS), where this difference in m/z was much smaller and therefore suggestive to be the result of Ca²⁺-binding to the S100A12 protein. The generated laser desorption/ionization-mass spectra also showed a smaller doublet peak with an m/z value of approximately 5,178.3 and a peak area that was exceeded by that of the major peak by a factor of about 4-6. Therefore, this additional minor peak can be considered to represent a non-specific, double charged monomer of canine S100A12.

The present invention provides the formation of a homodimer for canine S100A12 and was similar to the dimerization pattern shown for human S100A12 (with 2 Ca²⁺ ions per subunit) in the presence of >1 mM Ca²⁺. Homohexamers were, however, not revealed for the canine S100A12 protein but were for human S100A12 (with 3 Ca²⁺ ions per subunit) and proposed to be the extracellular form. A possible explanation for this discrepancy could be that, while the AA residues found to participate in coordinating the canonical Ca²⁺ ions within the EF-hand are highly conserved between human and canine S100A12, only one of the four residues shown to coordinate interdimer Ca²⁺ binding necessary for hexamer formation of human S100A12 exists in the AA sequence of canine S100A12 as predicted from cDNA analysis. These residues have been shown to vary widely among S100A12 from different species, and the existence of two lysines instead of Glu-55 and Gln-64, both of which appear to be necessary for hexamer formation of human S100A12, suggests a structural similarity of canine S100A12 with its bovine pendant, which has also been shown to lack a homohexameric form.

The weak nature of non-covalent macromolecular interactions renders them relatively fragile to the disruptive nature of SDS and desorption/ionization processes during SELDI-TOF MS analysis, thus resulting in their dissociation. In the presence as well as the absence of a strong reducing agent (DTT) during SDS-PAGE, canine S100A12 migrated to the same Mw position within the gel that is consistent with the monomeric form, whereas during native PAGE it was shown to migrate as a homodimer, which is consistent with results obtained for the human pendant and confirms our findings for canine S100A12 in a preparative PAGE experiment under native conditions. As has been reported for human S100A12, the formation of a homodimer was found to occur both in the presence as well as in the absence of Ca²⁺, indicating that Ca²⁺ is not essential for canine S100A12 dimerization. These findings suggest that the native state of canine S100A12 is dimeric. As in humans, porcines, and bovines but in contrast to rabbits, the AA sequence of the canine S100A12 monomer contains no cysteine residues based on cDNA analysis, which altogether indicate that the formation of such homodimeric structures results from a non-covalent association of canine S100A12 protein monomers. The existence of canine S100A12 as a non-covalently associated protein complex is further supported by the lack of evidence of any specific multimeric forms of the protein in SELDI-TOF-mass spectra, most likely due to their disruption during sample preparation and ion formation. Hence, while the use of this method is suitable to determine the size of the respective monomer, it is difficult as definitive evidence of homooligomerization and non-covalent interaction.

Homotetramerization of canine S100A12 in a buffer with a higher Ca²⁺ molarity (with or without micromolar amounts of Zn²⁺) was evident under native conditions in this study, and thus interpreted to be also non-covalent in nature. The formation of higher order oligomers in the presence of Ca²⁺ and Zn²⁺ has been reported in humans and porcines, and the HXXXH motif in the C-terminal region of the protein that has been suggested to participate in the binding of Zn²⁺ ions is also contained in the AA sequence of canine S100A12. In view of the different composition of the intracellular and the extracellular milieu, further experiments are warranted to determine whether the homodimeric, the monomeric, or a higher order oligomeric form of canine S100A12 occurs in vivo.

Immunologic cross-reactivity between different species serves as an indicator of high AA sequence similarity in the antigenic sites of a protein in these species. Given an unusually high sequence divergence of a protein belonging to the otherwise highly conserved S100-superfamily, as it has been reported for S100A12 from different species, a cross-reactivity with porcine serum but not with feline, equine, or bovine serum was unexpected. This finding leads the authors to speculate that porcine S100A12 may present the highest sequence similarity with canine S100A12 in the antigenic sites, although it is only conserved to 68.1% (as opposed to a 73.6, 72.5, and 70.3% AA sequence similarity of the feline, equine, and bovine pendant, respectively).

Similarities were found between canine, human, porcine, bovine, and rabbit S100A12 with regard to structural as well as biochemical properties (such as Mr and p1). It remains to be determined whether canine S100A12 occurs as a homodimer, a monomer, or a higher order oligomer in vivo. However, this does not have any effect on the use of immunoassays to assess canine S100A12 in biological fluids from dogs.

### Example 2

### Preparation of Polyclonal Antibodies

Polyclonal antisera against canine S100A12 were generated in two New Zealand White rabbits (Oryctolagus cuniculus). Both animals were repeatedly inoculated s.c. with pure canine S100A12 emulsified in complete (CFA) and incomplete Freund's adjuvant (IFA). The initial inoculation of both rabbits consisted of 200 μg canine S100A12 in CFA, and was followed by two booster injections with 150 μg canine S100A12 each in IFA every three weeks and another booster four weeks later. Due to an insufficient Ab response, one rabbit continued to receive four monthly boosters of 200 μg canine S100A12 in IFA s.c. into the prescapular nodal area. Ten days after each inoculation, blood samples were collected for evaluation of Ab titers using a simplified RIA and reactivity of the antisera was tested by radial double immunodiffusion as described (Heilmann et al., 2010). Anti-canine S100A12 antiserum from the rabbit that received four injections, collected following the third booster injection, was selected for the RIA with a final dilution of 1:12,000.

Polyclonal antibodies against canine S100A12 were purified by affinity chromatography using a chromatography column (HiTrap™ NHS-activated chromotography column, GE Healthcare, Uppsala, Sweden) prepared in accordance with the manufacturer's instructions. Following lipoprotein precipitation and buffer exchange to 75 mM Tris/HCl, 150 mM NaCl; pH 8.0, the antiserum was applied to the column. After a short incubation, antibodies were eluted with 100 mM glycine, 500 mM NaCl; pH 2.5, dialyzed against 25 mM Tris/HCl, 500 mM NaCl; pH 7.2 (BupH™ Tris buffered saline, Thermo Scientific, Rockford, Ill.), and stored at −80° C.

A fraction of the purified monospecific antibodies was dialyzed against 200 mM carbonate-bicarbonate; pH 9.4 (BupH™ Carbonate-Bicarbonate buffer, Thermo Scientific, Rockford, Ill.) and was coupled with horseradish peroxidase (HRP; EZ-Link® Plus Activated peroxidase, Thermo Scientific) according to the manufacturer's instructions. Following dialysis against 25 mM Tris/HCl, 150 mM NaCl; pH 8.0 the conjugate was purified using a Ni⁺ activated affinity chromatography column (Pierce® Conjugate purification kit, Thermo Scientific, Rockford, Ill.). After purification the buffer was exchanged against 100 mM sodium phosphate, 150 mM NaCl; pH 7.2 (BupH™ Phosphate buffered saline, Thermo Scientific), the antibody solution was mixed with two parts of a conjugate stabilizer solution (SuperFreeze® Conjugate stabilizer, Thermo Scientific) to preserve immunoreactivity and prevent freezing, and was stored at −20° C.

Immunologic cross-reactivity between canine, feline, bovine, porcine, ovine, caprine, equine, marmoset, meerkat, sea lion and penguin S100A12 was evaluated by precipitation of the respective serum against rabbit anti-canine S100A12 antiserum using radial double immunodiffusion (Ouchterlony test). The Ouchterlony test yielded a single strong precipitin line between rabbit anti-canine S100A12 antiserum and canine serum, extracts of fecal samples collected from dogs with acute hemorrhagic gastroenteritis, and porcine serum. However, no reactivity was found against feline, bovine, ovine, caprine, equine, marmoset, meerkat, sea lion and penguin serum. An Ouchterlony test was also performed for the purified canine S100A8 monomer and the canine S100A8/A9 complex (canine calprotectin), canine serum samples and canine fecal extracts (at a 1:5 dilution). No precipitation occurred between rabbit anti-canine S100A12 antiserum and purified canine S100A8 or canine S100A8/A9, respectively.

To demonstrate specificity of the rabbit anti-canine S100A12 antiserum, a modified Western blot was used to analyze the affinity of the antibody against canine serum samples (at a dilution of 1:20), canine fecal extracts (at a dilution of 1:500), the soluble cytosol fraction of leukocytes as well as a sample from each of the following purification stages, pure canine S100A12 in buffer D (positive control), and BSA (negative control). Immunodetection was performed as described above except that rabbit anti-canine S100A12 was used as primary antibody (primary antiserum dilution 1:2,000 in blocking buffer) and the secondary antibody (biotin-conjugated goat anti-rabbit IgG) was diluted 1:25,000 in blocking buffer. Western blot analysis using the anti-canine S100A12 antiserum clearly revealed one band at a M_{M} of approximately 7.8 kDa in samples from each of the purification stages, canine fecal extracts, and purified canine S100A12, whereas the rabbit anti-canine S100A12 pAb only weakly recognized canine S100A12 in canine serum samples. No bands were detected for BSA.

### Example 3

### Development and Validation of RIA

### I. Immunoassay Development

A. Production of the Tracer

A tracer was produced by labeling pure canine S100A12 with radioactive iodine (¹²⁵I) using the chloramine T method (Hunter et al., 1962). Briefly, 7.4 μg of previously purified canine S100A12 were dissolved in 0.25 M sodium phosphate (pH 7.5), and 7.4 μl Na¹²⁵I (0.74 mCi at the time of production) and 20 μg chloramine T were added. After 40 seconds incubating at approximately 23° C., 40 μg sodium metabisulfite and 1.72 mg potassium iodide were added to a final volume of 1 ml. ¹²⁵I-tagged canine S100A12 and free ¹²⁵I were separated by size exclusion and buffer-exchanged against 0.05 M sodium phosphate, 0.02% (w/v) NaN₃, 0.5% (w/v) BSA; pH 7.5 (RIAB). The ¹²⁵I-labeled canine S100A12 containing fractions were tested with the antiserum and selected based on a low non-specific binding (NSB, <1%) at a tracer-binding ratio for the zero standard (B₀/TC) of approximately 30% (Berson et al., 1968). Tracer was diluted in RIAB to approximately 30,000 cpm, and stored until further use during the RIA. Specific activity of the tracer was estimated (Chiang, 1987) and stability of ¹²⁵I-labeled canine S100A12 at different storage conditions investigated over 42 days (Law, 1996): aliquots of tracer were stored at +4 or −20° C. at approximately 0.2 or 2.3 mCi/L in polypropylene containers, or at 4° C. and approximately 0.2 mCi/L in non-inert glass containers.

B. Radioimmunoassay Development

The RIA was conducted in polypropylene tubes. Each tube received 100 μL canine S100A12 standard (fractional dilution from 200.0 to 0.2 μg/L), serum (1:20 diluted) or fecal extract (in a 1:8 or 1:160 dilution) in RIAB, 100 μl antiserum (diluted 1:4,000) in RIAB containing 0.05% polyoxyethylene sorbitan monolaurate and 10 mM EDTA, and 100 μL tracer. For the zero standard (B₀), the canine S100A12 standard was replaced by RIAB. NSB tubes received 200 μL RIAB with 0.025% polyoxyethylene sorbitan monolaurate and 5 mM EDTA, and 100 μL ¹²⁵I-labeled canine S100A12. Total count (TC) tubes contained 100 μL of tracer each. Following 4 hours of incubation at approximately 23° C., 100 μL rabbit carrier serum (diluted 1:100 in RIAB) and 1 mL secondary antibody solution were added to all tubes except TC, free fractions were separated from bound fractions by centrifugation for 30 min at 3,360×g and 4° C., and the remaining pellets were washed with RIAB. Tubes were analyzed using an automated γ-counter and software to calculate a 5PL-curve fit (y=f [x]=d+[(a−d)/(1+(x/c)^{b})^{e}]; y is the dependent variable, x is the independent variable, and a through e describe the shape of the curve) (Gottschalk et al., 2005). Standard concentrations of canine S100A12 (log scale) defined the abscissa, and ordinates were calculated as y=[(B_{std}/B₀)×100] (Midgley et al., 1969) where B_{std} is the NSB-subtracted cpm for the standard and B₀ the NSB-subtracted cpm obtained for B₀. Sample concentrations of canine S100A12 (in μg/L serum or ng/g feces) were determined by plotting the NSB-subtracted cpm against the standard curve. Samples with a canine S100A12 concentration beyond the standard range of the assay were further diluted and re-assayed. Aliquots of diluted fecal extracts with low, moderate, and high canine S100A12 concentrations were stored at −80° C., and served as quality controls.

### II. Immunoassay Analytical Validation

A. Collection and Processing of Serum and Fecal Specimens

Serum was obtained from 124 healthy dogs of various breeds and ages (median age: 4.5 years, range: 0.8-13.5 years). The protocol for collection of blood samples from healthy dogs was reviewed and approved by the Clinical Research Review Committee at Texas A&M University. Fecal samples were collected from 65 healthy dogs of various breeds and ages (median: 4.5 years, range: 0.8-11.1 years). All dogs were vaccinated, had been de-wormed regularly, and did not have any condition or receive any medications known to affect the gastrointestinal tract. Feces were collected, placed in pre-weighed polypropylene tubes, and immediately frozen until further use. Feces were then thawed and extracted using a previously established protocol (Heilmann et al., 2008). Fecal extracts were stored at −80° C. until assayed.

B. Radioimmunoassay Analytical Validation

The minimum detection limit of the RIA was determined by analyzing 20 duplicates of B₀ in the same assay run, and calculating the mean and SD. The canine S100A12 concentration that corresponded to the mean cpm minus three SD plotted against the standard curve, was defined as lower limit of detection of the RIA. The upper limit of the working range was determined by evaluating ten duplicates of a solution containing canine S100A12 in a concentration ten times the top standard in the same assay run (i.e., 2,000 μg/L), and calculating the mean and SD. The canine S100A12 concentration that corresponded to the mean cpm plus three SD was defined as maximum canine S100A12 concentration detectable by the RIA (upper limit of the working range). To determine linearity, accuracy, precision, and reproducibility of the RIA, samples with low, moderate, and high canine S100A12 concentrations were tested. Assay linearity was evaluated by dilutional parallelism for six different serum samples at serial twofold dilutions from 1:12-1:96. Linearity of the RIA for canine fecal extracts was tested using three different extracts at dilutions of 1:100-1:1,600, and three and two different extracts with a higher canine S100A12 concentration diluted 1:200-1:3, 200 and 1:400-1:6,400, respectively. Three different fecal extracts with low canine S100A12 concentrations were diluted 1:40-1:160. Linearity of the assay for cerebrospinal fluid (CSF) was tested with a CSF specimen at a serial twofold dilution from 1:10-1:1,280. Assay accuracy was tested by spiking seven different sera and seven different fecal extracts with known concentrations of canine S100A12 (i.e., 0, 0.2, 1, 2, 5, 10, 20, 50, and 100 μg/L for serum samples and 0, 200, 800, 1,600, 4,000, 8,000, 16,000, 40,000 and 80,000 ng/g for fecal extracts). The percentage of standard antigen recovery was calculated as [observed value (μg/L or ng/g)/expected value (μg/L or ng/g)]×100. Precision of the assay was evaluated by assaying seven different serum samples and seven different fecal extracts from dogs ten times within the same assay followed by calculating the intra-assay coefficients of variation (% CV=[SD/mean]×100). Reproducibility of the assay was determined by analyzing seven different canine sera and seven different canine fecal extracts in ten consecutive assay runs and calculating inter-assay % CVs. Analytical specificity was determined by assessing cross-reactivity of the RIA with 0.2-20,000 μg/L canine S100A8/A9 (canine calprotectin), the closest structural analogues of canine S100A12 available. Species specificity of the RIA was tested by evaluating two different porcine sera at dilutions of 1:10-1:80.

C. Assessment of Interference with the RIA Due to Hyperlipidemia

Endogenous interference of increased concentrations of lipids as common sample matrix components with the assay was tested by spiking four different canine serum samples (with low, moderate, and high canine S100A12 concentrations) with known concentrations of a commercially available intravenous lipid formulation (i.e., 0, 100, 250, 500, 750, 1,100, 1,250, 1,500, 1,750, 2,000, and 3,000 mg/dL) followed by a Friedman test for data analysis. Also, concentrations of canine S100A12 were measured in 32 grossly lipemic serum samples before and after high speed centrifugation at 16,000×g for 15 min followed by a Wilcoxon signed rank test for paired data analysis.

D. Reference Interval for Serum Canine S100A12

A reference interval for serum canine S100A12 was established by evaluating serum samples from 124 healthy adult dogs and calculating the central 95th percentile. Serum canine S100A12 concentrations were compared between healthy male and female dogs using a Mann-Whitney U test, and between dogs of different age groups (<2, 2-4,4-6, 6-8, and >8 years) by a Kruskal-Wallis test.

E. Reference Interval and Intra-Individual Variation of Fecal Canine S100A12

Distribution of canine S100A12 in feces was assessed by determining the variation within a single defecation for 12 dogs. Three random aliquots of approximately 1.0 g were collected from different portions of feces (spot samples) and extracted as described. The remainder of the feces was diluted 1:1 in extraction buffer, homogenized, and two aliquots (homogenates) were sampled and extracted with a final dilution of 1:5. Variability of fecal canine S100A12 concentrations was evaluated for each dog by calculating the % CV for canine S100A12 quantified in all extracts. Short-term intra-individual variation of fecal canine S100A12 over five consecutive days was evaluated for 40 of the 65 healthy dogs. To determine a reliable and efficient sampling strategy, mean fecal canine S100A12 concentrations and intra-individual variation (i.e., CV) were evaluated for various numbers of sampling days (day 1; days 1 and 2; days 1-3; days 1-4; and days 1-5) by use of a Friedman test with a Dunn's post hoc test. A reference interval for fecal canine S100A12 concentration was established from the central 95th percentile of the mean fecal canine S100A12 concentrations of three samples (collected on days 1-3) from each of the 65 healthy dogs. Mean fecal canine S100A12 concentrations of three sampling days were compared between healthy dogs of different age groups (<3, 3-6, and >6 years) using a Kruskal-Wallis test.

F. Biologic Variation of Canine S100A12 in Serum

Twelve apparently healthy pet dogs of different breeds (7 females and 4 males; median age: 3 years, range: 2-8 years) were enrolled into the study, the protocol for which had been reviewed and approved by the Institutional Animal Care and Use Committee at Oregon State University and has been detailed elsewhere (Carney et al., 2011). Briefly, biologic variation of serum canine S100A12 concentrations was evaluated over a period of 2.6 months in 11 of the dogs; one dog was excluded from the study due to the development of inflammatory skin disease immediately following the sample collection period. From each dog, blood was collected by venipuncture daily for seven days, weekly for six weeks, and a final sample one month later. Immediately after collection, serum was separated from the samples followed by storage at −80° C. until analysis.

Serum canine S100A12 was measured simultaneously in all specimens using standard assay criteria (i.e., samples that yielded a replicate CV of ≧15% were re-assayed). Samples were analyzed over five assay runs ensuring use of the same lots of reagents, standards, and quality controls. To eliminate between-run analytical variation, serial samples from the same individual were assayed in the same RIA. Tests for outliers were carried out at three levels (i.e., within-run analytical variance, and intra- and inter-individual variation), and a nested analysis of variance (ANOVA) model was used to calculate analytical and biological components of variation (Fraser et al., 1989): analytical (CV_{A}), intra-individual (CV_{I}), inter-individual (CV_{G}), and total variation (CV_{T}) (Fraser et al., 1989; Wu et al., 2009). Indices of biological variation were expressed as index of individuality (II; an II of ≦0.6 would indicate that individuals tend to be distinct from each other and thus, use of a population-based reference interval would not be appropriate whereas an II of ≧1.4 suggests a population-based reference interval may be useful), index of heterogeneity (IH; the ratio of CV_{I} to the theoretical CV), and minimum critical difference (MCD_{0.05}; the percentage difference in concentration that is significant at p≦0.05 and is calculated using the 90th percentile of the observed distribution of within-subject variances) (Fraser et al., 1989; Wu et al., 2009).

Assumptions of normality and equality of variances were tested using a Shapiro-Wilk W test and a Bartlett's test, respectively. Microsoft Excel, GraphPad Prism or JMP software were used for all calculations and statistical analyses.

### III. Results

A. Radioimmunoassay development and analytical validation

Immunization with pure canine S100A12 yielded a moderate titer of anti-canine S100A12 Ab in both rabbits. Specific activity of the tracer was 636.4±80.8 Ci/mmol (mean±SD). ¹²⁵I-labeled canine S100A12 was least stable when stored at −20° C. and approximately 2.3 mCi/l. For optimal stability, the tracer was stored in polypropylene tubes at −20° C. and a radiochemical concentration of approximately 0.2 mCi/L, and yielded a good performance of the RIA up to 42 days without any effect on NSB.

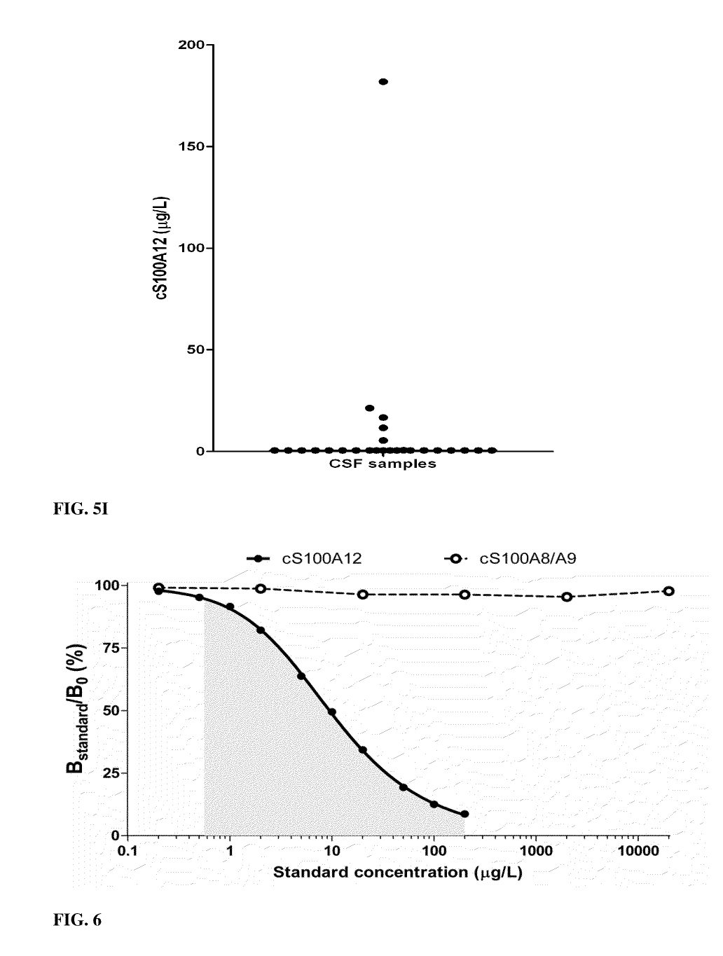

The assay was linear from 1-100 μg/L and had a working range between 0.6 and 432.7 μg/L (FIG. 6). Thus, for serum and fecal samples assayed in a 1:20 and 1:800 [1:40] dilution, respectively, the minimum detection limit of the assay was 11.2 μg/L and 500 ng/g [24 ng/g], respectively, with a maximum detection limit of 8,654.4 μg/L and 346,200 ng/g [17,400 ng/g], respectively. No cross-reactivity was observed with the canine S100A8/A9 protein complex up to 20,000 μg/L (FIG. 6). Dose-response curves for the serial dilution of serum and fecal samples with a wide range of canine S100A12 concentrations and of one CSF specimen paralleled the standard curve (Table 3), and expected and observed values for spiking recovery of the assay were closely correlated (Table 4). For precision and reproducibility testing of the RIA, mean values and intra- and inter-assay CVs are presented in Table 5. Aliquots of canine S100A12 standard solutions were stored at −80° C., and no change in the standards was observed after storage of 18 months. Spiking of sera with various concentrations of lipid components did not affect the results (p=0.104) whereas canine S100A12 concentrations (median: 201.2 μg/L) were significantly decreased after high speed centrifugation (median: 138.1 μg/L; p<0.0001). By use of this RIA, S100A12 was detected in one of two porcine sera, the serial dilution of which yielded observed-to-expected ratios of 103.2±16.4% (mean±SD).

TABLE 3
Results for dilutional parallelism of serum samples and fecal
extracts and of one CSF specimen for the newly developed
canine S100A12 RIA. Observed-to-expected (O/E) ratios for the
serial dilution of six serum samples ranged from 97.2-146.8%
(mean ± SD: 122.5 ± 14.9%) and of 11 fecal extracts from
75.3-129.8% (104.8 ± 12.5%). The O/E ratios for the serial
dilution of one CSF sample ranged from 77.2-116.5 (91.2 ± 13.4%).

| | | canine | |
|---|---|---|---|
| | | S100A12 | Observed/ |
| Specimen | Dilutions | (μg/L or ng/g) | expected ±SD (%) |
| Serum 1 | 1:2-1:16 | 181.7 | 123.1 ± 16.2 |
| Serum 2 | 1:2-1:16 | 191.6 | 123.8 ± 8.5 |
| Serum 3 | 1:2-1:16 | 197.0 | 117.7 ± 16.5 |
| Serum 4 | 1:2-1:16 | 217.6 | 132.1 ± 17.5 |
| Serum 5 | 1:2-1:16 | 312.6 | 133.5 ± 9.5 |
| Serum 6 | 1:2-1:16 | 335.3 | 104.5 ± 7.1 |
| Fecal 1 | 1:40-1:160 | 186 | 104.4 ± 24.3 |
| Fecal 2 | 1:40-1:160 | 648 | 95.3 ± 15.0 |
| Fecal 3 | 1:40-1:160 | 1,887 | 112.7 ± 3.5 |
| Fecal 4 | 1:100-1:1,600 | 612 | 109.6 ± 14.6 |
| Fecal 5 | 1:100-1:1,600 | 4,127 | 107.5 ± 18.3 |
| Fecal 6 | 1:100-1:1,600 | 14,572 | 100.9 ± 15.0 |
| Fecal 7 | 1:200-1:3,200 | 2,228 | 97.1 ± 16.0 |
| Fecal 8 | 1:200-1:3,200 | 5,043 | 101.9 ± 8.8 |
| Fecal 9 | 1:200-1:3,200 | 12,159 | 110.0 ± 5.6 |
| Fecal 10 | 1:400-1:6,400 | 48,434 | 101.8 ± 5.2 |
| Fecal 11 | 1:400-1:6,400 | 688,947 | 115.8 ± 9.3 |
| CSF 1 | 1:10-1:1,280 | 736.9 | 91.2 ± 13.4 |

SD: standard deviation

TABLE 4
Results for spiking recovery of canine S100A12 as determined by
the newly developed RIA. Observed-to-expected ratios obtained
by spiking seven canine serum samples and seven fecal extracts
with eight different concentrations of canine S100A12 ranged
from 87.8 to 130.4% (mean ± SD: 100.6 ± 6.5%)
and from 84.8 to 143.8% (103.7 ± 10.6%), respectively.

| | canine | |
|---|---|---|
| | S100A12 | Observed/ |
| Specimen | (μg/L or ng/g) | expected ±SD (%) |
| Serum 1 | 83.5 | 97.1 ± 3.9 |
| Serum 2 | 103.1 | 97.2 ± 5.0 |
| Serum 3 | 113.2 | 98.0 ± 4.2 |
| Serum 4 | 187.3 | 100.6 ± 3.2 |
| Serum 5 | 402.3 | 104.0 ± 3.3 |
| Serum 6 | 442.0 | 99.7 ± 6.2 |
| Serum 7 | 772.2 | 107.7 ± 10.4 |
| Fecal 1 | 6,623 | 97.4 ± 6.2 |
| Fecal 2 | 6,935 | 105.1 ± 4.8 |
| Fecal 3 | 8,054 | 109.6 ± 7.7 |
| Fecal 4 | 14,139 | 94.6 ± 6.7 |
| Fecal 5 | 24,847 | 99.7 ± 4.1 |
| Fecal 6 | 71,045 | 113.8 ± 15.3 |
| Fecal 7 | 146,089 | 106.1 ± 12.0 |

SD: standard deviation

TABLE 5
Precision and reproducibility of the RIA for canine S100A12.

| | Serum | Mean | CV | Fecal | Mean | CV |
|---|---|---|---|---|---|---|
| | sample | (μg/L) | (%) | sample | (ng/g) | (%) |
| Intra-assay | 1 | 196.5 | 1.5 | 1 | 5,236 | 3.8 |
| variability | 2 | 204.1 | 8.1 | 2 | 6,728 | 5.5 |
| | 3 | 224.0 | 6.3 | 3 | 7,299 | 3.8 |
| | 4 | 342.1 | 4.5 | 4 | 18,900 | 4.2 |
| | 5 | 437.5 | 6.6 | 5 | 79,720 | 6.1 |
| | 6 | 713.0 | 5.7 | 6 | 189,851 | 7.4 |
| | 7 | 2,021.3 | 6.2 | 7 | 215,221 | 7.8 |
| Inter-assay | 1 | 169.2 | 5.6 | 1 | 6,977 | 5.3 |
| variability | 2 | 246.4 | 5.9 | 2 | 7,684 | 5.9 |
| | 3 | 265.5 | 7.8 | 3 | 10,190 | 5.7 |
| | 4 | 272.9 | 5.8 | 4 | 12,694 | 4.0 |
| | 5 | 299.2 | 5.1 | 5 | 26,025 | 2.9 |
| | 6 | 394.3 | 3.3 | 6 | 92,566 | 7.5 |
| | 7 | 463.7 | 6.5 | 7 | 228,744 | 8.7 |

CV: coefficient of variation

B. Biological Variation and Reference Interval for Serum Canine S100A12

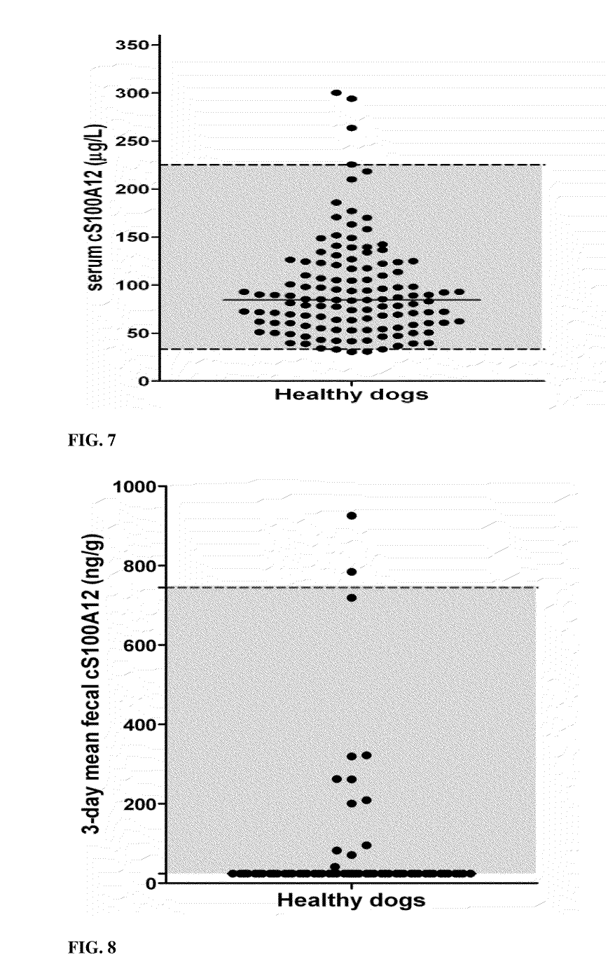

Serum canine S100A12 concentrations in samples from 124 healthy pet dogs ranged from 30.4-300.2 μg/L (median: 84.4 μg/L) and the reference interval for serum canine S100A12 concentration was established as 33.2-225.1 μg/L (FIG. 7). Serum canine S100A12 concentrations were not significantly different (p=0.819) between healthy male and female dogs. Although the number of sexually intact male (n=15) and female (n=13) dogs was small compared to neutered males (n=40) and females (n=56), serum canine S100A12 concentrations were also not significantly different among those four groups (p=0.068). Also, serum canine S100A12 concentrations were not significantly different among healthy dogs of different age groups (p=0.427).

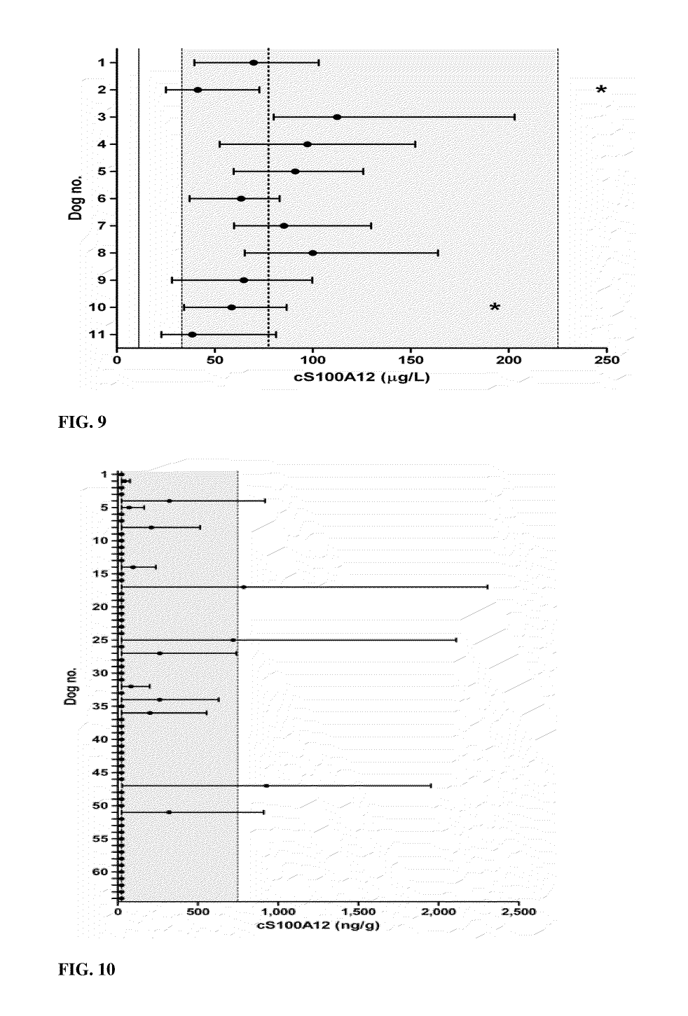

For evaluation of the biological variability, a total of 14 serial specimens were collected from 9 dogs and 13 serial samples from 2 dogs. Two within-subject outliers were detected (one each from dogs 2 and 10) and excluded from further analysis, yielding a total of 150 serum samples (FIG. 9) and slightly right-skewed data. No outlying observations (maximum variance/sum of the variances=0.086; Cochrane test) or outliers among mean concentrations of subjects (extreme minus next highest concentration=15% of the concentration range; Reed's criterion) were detected. CV_{A} was calculated as 5.7%, CV_{I} as 29.2%, and CV_{G} as 31.2% resulting in a CV_{T} of 66.0%. Index of Individuality (II) was determined to be 0.95 and IH was 56.4, yielding a one-sided MCD_{0.05} of 84.9%.

C. Intra-Individual Variability and Reference Interval for Fecal Canine S100A12

For canine S100A12 concentrations in three fecal spot samples collected from one single defecation, CVs ranged from 0.0 to 152.9% (mean±SD: 34.7±53.9%). Fecal canine S100A12 in three samples from each of the 65 healthy dogs ranged from <24 to 2,305 ng/g (median: <24 ng/g), with three-day sample mean canine S100A12 concentrations ranging from <24 to 926 ng/g (median: <24 ng/g) (FIG. 10). In five consecutive fecal samples from 40 dogs, fecal canine S100A12 ranged from <24 to 5,686 ng/g (median: <24 ng/g) and the five-day sample mean fecal canine S100A12 from <24 to 1,381 ng/g (median: <24 ng/g). Mean fecal canine S100A12 concentrations were not significantly different (p=0.581) among different numbers of sampling days; significance was reached for maximum fecal canine S100A12 concentrations and CVs overall (both p<0.001), but not in any of the post hoc tests. The reference interval for fecal canine S100A12 concentration was established as a three-day sample mean of <24 to 745 ng/g (FIG. 8). Three-sample mean fecal canine S100A12 concentrations were not significantly different among healthy dogs of different age groups (p=0.410) or between healthy male and female dogs (p=0.464).

### IV. Discussion

A radioimmunoassay for the measurement of canine S100A12 in serum samples and fecal extracts was successfully established. The minimum detection limit of the assay was calculated to be 11.2 μg/L for serum and 24 ng/g feces for fecal samples, respectively, which appears to be adequate considering the upper limit of the reference intervals for serum (225.1 μg/L) and fecal samples (745 ng/g), and the intent to identify dogs with increased serum and/or fecal canine S100A12 concentrations. Both a very dilute antiserum and tracer were chosen to increase the sensitivity of the assay and allow measuring canine S100A12 in fecal samples from healthy dogs and dogs with gastrointestinal inflammation, assumed to have increased fecal canine S100A12 concentrations. This decreases the range of binding ratios (B_{std}/B₀) but appeared to not affect the overall assay performance. However, as fecal canine S100A12 concentrations were below the minimum detection limit of the RIA (24 ng/g) in 51/65 healthy dogs (78.5%), further studies in canine patients with gastrointestinal inflammation will need to be conducted to show whether this assay is sufficiently sensitive for detection of canine S100A12 in fecal samples from clinical patients.

Dilutional parallelism and spiking recovery of canine S100A12 in canine sera and fecal extracts as well as in CSF indicate linearity and accuracy of the RIA, and the intra- and inter-assay CVs demonstrate precision and reproducibility of the assay. Compared to reports in humans (Larsen et al., 2007), concentrations of serum canine S100A12 did not change after repeated freeze-thaw cycles in our study (data not shown), and attempts to analyze serum and fecal samples using RIAB devoid of EDTA yielded a matrix effect rendering quantification of canine S100A12 impossible. Based on the signal (i.e., cpm) obtained and the fact that this was not seen in canine S100A12 standards, whereas spiking canine S100A12 standards into an “analyte-free” matrix consisting of feline serum samples and fecal extracts (shown to not cross-react using this assay) yielded the same effect, we speculate that ¹²⁵I-canine S100A12 was bound to matrix components (possibly forming multimers with the native protein in specimens) and that EDTA in a concentration expected to chelate most of the Ca²⁺ in serum or extraction buffer (Ca²⁺ was added to increase the stability of S100 proteins) effectively neutralized this effect. However, an increased interaction of canine S100A12 with the surface of the assay tubes (presumably due to its increased hydrophobicity upon Ca²⁺ binding) also needs to be considered.

Difficulties raising anti-canine S100A12 antiserum in rabbits using a common vaccination protocol for producing pAb were unexpected and have not been reported for human S100A12. Similar difficulties were experienced when generating pAb against canine S100A8/A9 in rabbits (Heilmann et al., 2008); however, S100A12 is reported to be less well conserved among species than S100A8/A9 (Moroz et al., 2003). As for canine S100A8/A9, modifying both the amount of antigen inoculated and injection site led to an increase in anti-canine S100A12 titer. Thus, the insufficient pAb response against canine S100A12 in one rabbit may be due to a high epitopic homology between canine and rabbit S100A12, the size of canine S100A12 being close to the minimum for immunogens (˜5 kDa), or idiosyncrasy. However, sequence homology of rabbit S100A12 with the canine (63%) and human protein (67%) is similar, and canine S100A12 is assumed to form homooligomeric complexes in the Ca²⁺-containing buffer used for the inoculations (Heilmann et al., 2010).

Lipids represent matrix components that commonly interfere with immunoassay performance. While the spiking of samples with lipid components in concentrations measured in patients with severe hypertriglyceridemia/hyperlipidemia did not affect the results in our study, separating the majority of the lipid fraction from the sera by high speed centrifugation appeared to also remove a significant portion of canine S100A12 from the samples. It seems plausible that a fraction of canine S100A12 is found in the lipid portion of the samples (possibly due to its hydrophobicity at Ca²⁺ concentrations found in serum). Therefore, in one embodiment the lipemic serum samples can be assayed or an aliquot removed before separating the lipid fraction.

Concentrations of canine S100A12 in feces from healthy dogs varied, which may be due to a patchy distribution of canine S100A12-expressing cells within the gastrointestinal mucosa and/or variations in gastrointestinal passage leading to variation in the concentration of fecal proteins, and has also been shown for other fecal markers in dogs (Steiner et al., 2003; Heilmann et al., 2008). Therefore in one embodiment, fecal samples to be analyzed can be collected on three consecutive days.

Fecal canine S100A12 concentrations were not significantly different among pet dogs of various age groups, which may suggest that, in contrast to humans (de Jong et al., 2006; Kaiser et al., 2007; Sidler et al., 2008), age related changes do not occur.

Detection of porcine S100A12 by this assay was expected as cross-reactivity between canine S100A12 and porcine S100A12 has been shown previously (Heilmann et al., 2010). Moreover, linearity of the RIA for porcine serum samples indicates a potential application of this assay for S100A12 quantification in this species.

Biological variation is determined by pre-analytic factors associated with the collection of samples and true biological variance, whereas analytical variance arises from the methodology, instrumentation, and technical skills (Fraser et al., 1989). The protocol that was used for collection and analysis of samples to determine the biological variation has the advantage of eliminating long-term (i.e., inter-assay) analytical variation, thus rendering intra-assay variability (CV_{A}; estimated from analyzing duplicates of samples) the only component of analytical variation. The analytical goal of CV_{A}≦½×CV_{I} (Fraser et al., 1989) has also been satisfied in this study. Although serum canine S100A12 appears to be maintained within a relatively narrow concentration window in individual dogs, it showed moderate individuality and, as indicated by the MCD_{0.05}, moderate changes in serum canine S100A12 between sequential measurements in a dog are necessary to be considered relevant rather than reflecting biological and/or analytical variation. Thus, the use of a conventional population-based reference interval to detect increased canine S100A12 concentrations may or may not be appropriate, and needs to be further evaluated in dogs with acute and chronic inflammatory diseases. Interestingly, using the MCD_{0.05} with the median (84.4 μg/1) and the 75th percentile determined for serum canine S100A12 concentrations in the reference sample group yielded canine S100A12 concentrations coinciding with the upper limit of the central 90th percentile and the upper limit of the reference interval calculated (225.1 μg/1), respectively. This demonstrates that the reference interval established is within reasonable limits.

In summary, the RIA described here is sensitive, linear, accurate, precise, and reproducible, and has the potential to distinguish healthy dogs from dogs with inflammatory diseases, assumed to be associated with increased serum and/or fecal canine S100A12 concentrations.

### Example 4

### Development and validation of ELISA

An enzyme-linked immunosorbent assay (ELISA) was developed and analytically validated for the measurement of canine S100A12 in bodily fluids (e.g., serum/plasma, cerebrospinal fluid, or synovial fluid) and excretions (e.g., urine or feces) from dogs.

### I. Collection and Processing of Fecal Specimens

Fecal samples were collected from 53 healthy adult pet dogs of various breeds and ages (median age: 4.5 years, range: 0.8-11.1 years). All dogs were vaccinated, had been de-wormed regularly, and did not have any condition or receive any medications known to affect the gastrointestinal tract. Feces were collected, placed in pre-weighed polypropylene tubes, and immediately frozen until further use. Feces were then thawed and extracted using a previously established protocol. Fecal extracts were stored at −80° C. until assayed.

II. Development of a Sandwich ELISA for Fecal Canine S100A12 Measurement 96-well flat-bottom enhanced binding ELISA plates (MaxiSorp™ Nunc-Immuno™ Plates, Thermo Scientific, Rockford, Ill.) were coated with the affinity-purified polyclonal anti-canine S100A12 antibody (200 ng/well) in 200 mM carbonate-bicarbonate; pH 9.4, incubated for 1 h at 37° C., and washed three times with 25 mM Tris/HCl, 150 mM NaCl, 0.05% (v/v) polyoxyethylene-20 sorbitan monolaurate; pH 8.0 (wash buffer). Nonspecific binding sites were blocked with 25 mM Tris/HCl, 150 mM NaCl, 0.05% (v/v) polyoxyethylene-20 sorbitan monolaurate, 10% (w/v) bovine serum albumin (BSA); pH 8.0, and plates incubated for 1 h at 37° C. and again washed three times with wash buffer. Plates were kept at 4° C. short-term (i.e., up to three days) until used for an assay.

Standard solutions, blanks, three quality controls (QC) with different canine S100A12 concentrations, and test samples were applied to each plate in duplicates of 100 μL solution each. Standard solutions (5, 2, 1, 0.5, 0.2, 0.1, and 0.02 ng/mL), QCs and test samples (both assayed in a 1:100 dilution) were prepared in 25 mM Tris/HCl, 150 mM NaCl, 0.05% (v/v) polyoxyethylene-20 sorbitan monolaurate, 0.5% (w/v) BSA; pH 8.0 (assay buffer). For the blanks, the canine S100A12 standard was replaced by assay buffer. Test samples with a canine S100A12 concentration beyond the standard range of the assay were diluted 10-fold and re-assayed. Plates were incubated again for 1 h at 37° C. and washed three times as described. To detect captured antigen, plates were incubated with the HRP-conjugated antibody diluted in assay buffer (15 ng/well) for another hour at 37° C., washed three times, and developed with a stabilized 3,3′,5,5′-tetramethyl benzidine (TMB) substrate (1-Step™ Ultra TMB-ELISA, Thermo Scientific). After 5 min incubation, the reaction was stopped by adding 4 M acetic acid, 0.5 M sulfuric acid, and the absorbance was measured in each well at 450 nm using an automated plate reader (Synergy 2 Alpha Microplate Reader, BioTek®, Winooski, Vt.). A commercial software (Gen5™ Data Analysis Software (v1.05), BioTek®, Winooski, Vt.) was used to calculate a 5-parameter logistic curve fit (y=f[x]=d+[(a−d)/(1+(x/c)^{b})^{e}], where y is the dependent variable, x is the independent variable, and a through e describe the shape of the curve) and to determine canine S100A12 concentrations in test samples. To optimize the assay several parameters were analyzed during the development phase; different concentrations of primary (capture) and secondary (conjugate) antibodies as well as shaking versus non-shaking incubation at 37° C. were compared.

### III. Analytical Validation of the ELISA

The immunoassay was analytically validated by determination of the lower detection limit, dilutional parallelism, spiking recovery, and intra- and inter-assay variability. Lower detection limit of the assay was determined by calculating the mean response plus three standard deviations (SD) for 20 replicates of the blank solution and transposing this value onto the standard curve. Dilutional parallelism was determined by evaluating three fecal samples at a serial twofold dilution from 1:100-1:1,600 and two samples with a higher canine S100A12 concentration at serial twofold dilutions from 1:400-1:6,400. The remaining validation parameters were determined using four different fecal extracts in a final dilution of 1:100 with assay buffer. Spiking recovery was determined by adding 0, 0.05, 0.1, 0.2, 0.5, 1.0, and 2.5 ng/mL purified canine S100A12 to each of the four test samples and calculating the percentage of standard antigen recovery ([observed value (ng/g)/expected value (ng/g)]×100). Intra-assay variability of the assay was evaluated by assaying the four fecal extracts 10 times each within the same assay and calculating the intra-assay coefficients of variation for each sample (% CV=[SD/mean]×100). Inter-assay variability of the assay was determined by analyzing the four fecal extracts in 10 consecutive assay runs and calculating inter-assay % CVs. A reference interval for fecal canine S100A12 was established from the central 95th percentile of three-day mean fecal canine S100A12 concentrations measured in 53 healthy pet dogs.

### IV. Agreement to the Canine S100A12 RIA

Agreement between results obtained by the newly developed canine S100A12 ELISA and those obtained from the previously developed canine S100A12 RIA was evaluated by using 62 canine fecal samples that were previously assayed and measured beyond the lower detection limit of the canine S100A12 RIA, and re-testing those samples on the newly developed ELISA. Fecal canine S100A12 concentrations measured by the ELISA were plotted against those measured using the RIA, followed by calculation of a Spearman correlation coefficient ρ and a Passing-Bablok regression. All 62 fecal samples were then analyzed to assess any bias observed between the two assay methods. Normality of the differences was evaluated using a Shapiro-Wilk W test.

### V. Results

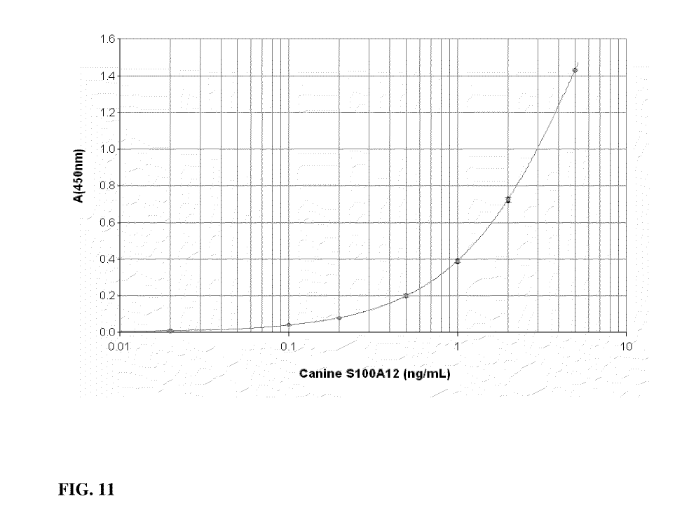

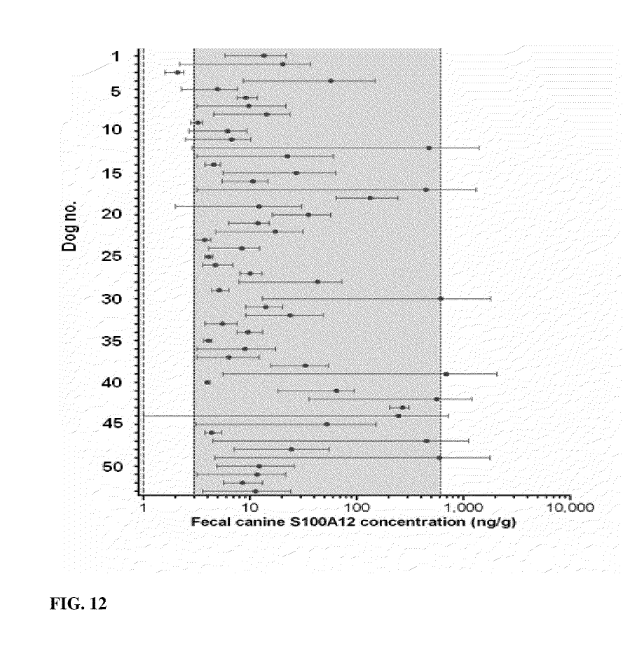

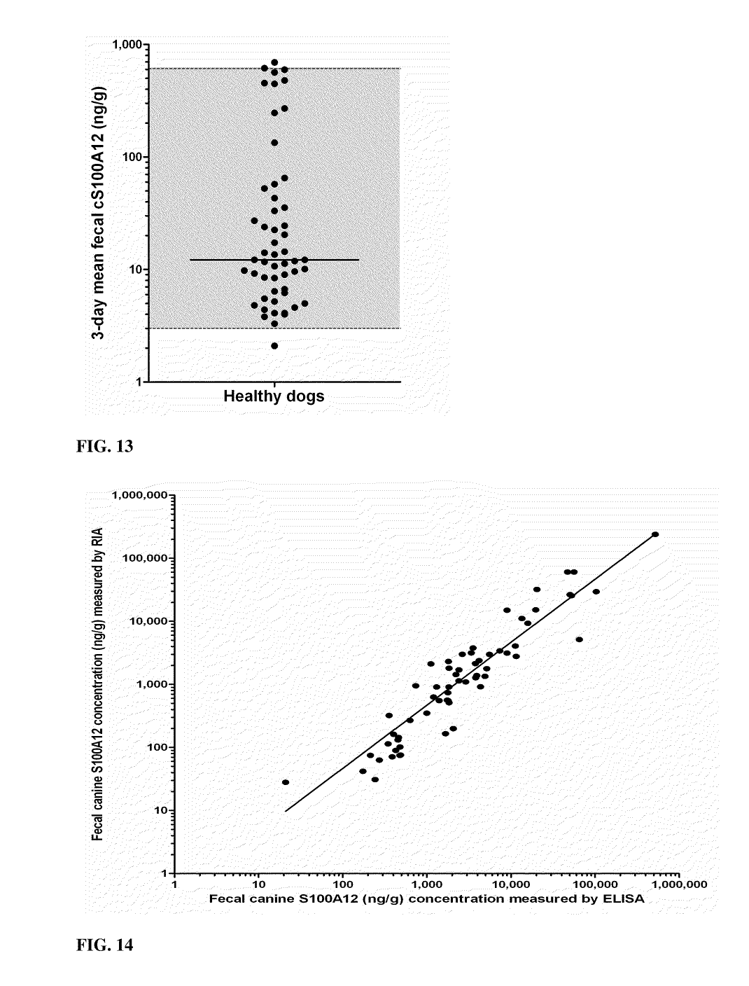

An ELISA for measuring canine S100A12 in fecal samples was successfully established and yielded reproducible standard curves (FIG. 11). The lower detection limit of the assay was 0.010 ng/mL, which considering the 1:100 dilution used for fecal samples, translates into a fecal canine S100A12 concentration of 1 ng/g. Observed-to-expected (O/E) ratios for dilutional parallelism of five fecal extracts ranged from 77.3 to 116.9% (mean±SD: 95.8±10.1%) (Table 6), and O/E ratios for spiking recovery of four fecal samples from 76.3 to 103.9% (mean±SD: 91.3±7.3%) (Table 7). Coefficients of variation for intra- and inter-assay variability were <4.2% and ≦10.9%, respectively (Table 8). Fecal canine S100A12 concentrations in healthy dogs ranged from <1-2,067 ng/g (median: 8 ng/g) (FIG. 12) with three-day mean canine S100A12 concentrations ranging from 2-694 ng/g (median: 12 ng/g). The reference interval for fecal canine S100A12 was calculated as a three-day sample mean of 3-610 ng/g (FIG. 13). Paired canine S100A12 concentrations in 62 fecal samples that were assayed using both the newly developed ELISA and the previously developed RIA were correlated (FIG. 14) as demonstrated by a Spearman ρ of 0.9309 (95% CI: 0.8858-0.9586). A linear relationship was confirmed for results obtained by ELISA and RIA, and a Passing-Bablok test identified a constant (α=0.8916; 95% CI: 0.6389-1.1443) and a proportional bias (β=0.8262; 95% CI: 0.7468-0.9055) between the two methods. For the 62 canine fecal samples, the difference in canine S100A12 concentrations between both methods was plotted against their mean (Bland-Altman plot; FIG. 15) following a common log transformation as the differences were shown to be related to the mean (p<0.0001).

TABLE 6
Results for dilutional parallelism of fecal extracts using the newly
developed canine S100A12 ELISA. Observed-to-expected ratios for the
serial two-fold dilution of five fecal extracts ranged from 77.3 to 116.9%
(mean ± SD: 95.8 ± 10.1%).

| | | canine | Observed/ |
|---|---|---|---|
| Specimen | Dilutions | S100A12 (ng/g) | expected ±SD (%) |
| Fecal 1 | 1:100-1:1,600 | 122.3 | 105.6 ± 14.2 |
| Fecal 2 | 1:100-1:1,600 | 160.5 | 94.7 ± 11.6 |
| Fecal 3 | 1:100-1:1,600 | 528.7 | 89.1 ± 6.7 |
| Fecal 4 | 1:400-1:6,400 | 932.4 | 95.3 ± 1.2 |
| Fecal 5 | 1:400-1:6,400 | 1,742.4 | 91.4 ± 4.8 |

SD: standard deviation

TABLE 7
Results for spiking recovery of canine S100A12 in fecal extracts as
determined by the new ELISA. Observed-to-expected ratios spiking
four different fecal extracts with six different canine S100A12
concentrations ranged from 76.3 to 103.9% (91.3 ± 7.3%).

| | canine S100A12 | Observed/ |
|---|---|---|
| Specimen | (ng/g) | expected ±SD (%) |
| Fecal 1 | 320 | 87.2 ± 8.5 |
| Fecal 2 | 557 | 88.6 ± 7.7 |
| Fecal 3 | 883 | 95.2 ± 6.0 |
| Fecal 4 | 2,656 | 94.4 ± 4.2 |

SD: standard deviation

TABLE 8
Precision and reproducibility of the RIA for canine S100A12.

| | Serum | Mean | CV |
|---|---|---|---|
| | sample | (ng/g) | (%) |
| Intra-assay | 1 | 277.8 | 3.5 |
| Variability | 2 | 671.8 | 3.9 |
| | 3 | 1,002.6 | 4.2 |
| | 4 | 1,613.3 | 3.7 |
| Inter-assay | 1 | 116.6 | 10.9 |
| Variability | 2 | 395.5 | 6.9 |
| | 3 | 1,052.0 | 4.7 |
| | 4 | 1,879.2 | 5.8 |

CV: coefficient of variation

### VI. Discussion and Conclusions

The assay was linear, accurate, precise, and reproducible, and its analytical sensitivity was superior to that of the previously developed RIA and yielded a concentration above the minimum detection limit of the assay for almost all of the samples (158/159; 99.4%) collected from healthy dogs. The reference interval for fecal canine S100A12 concentration using this newly established ELISA was determined as 3-610 ng/g.

### Example 5

### Analysis of S100A12 in Biological Samples

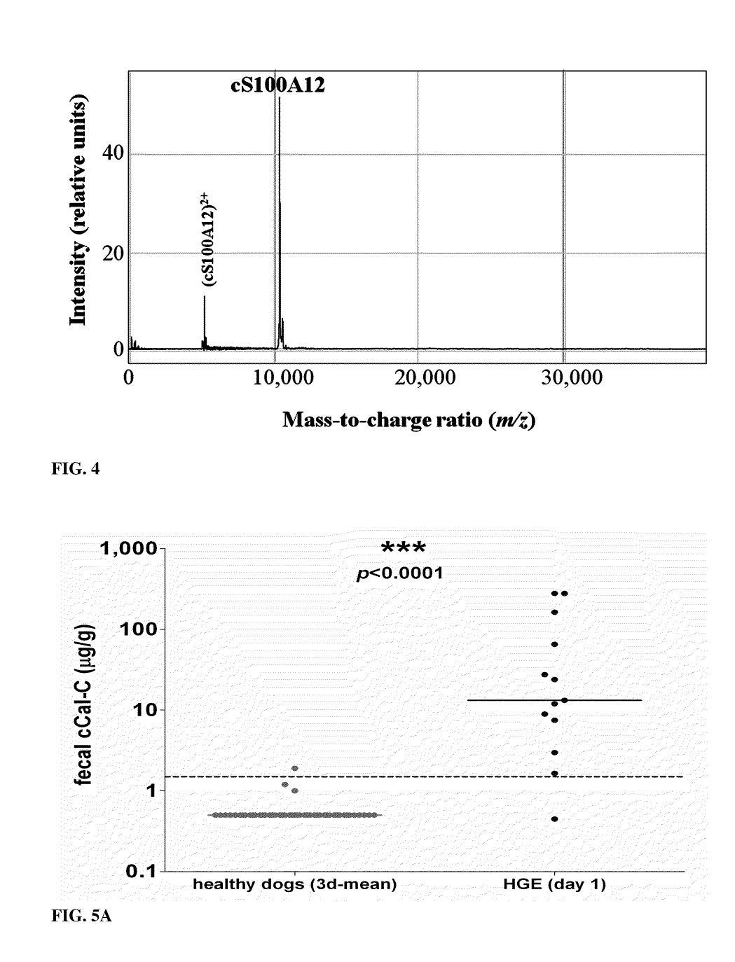

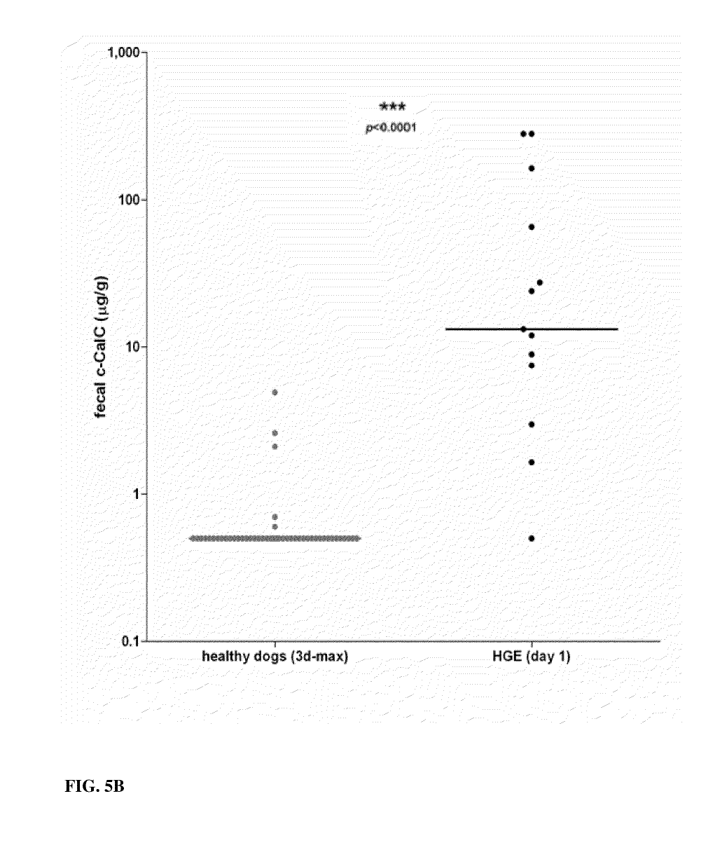

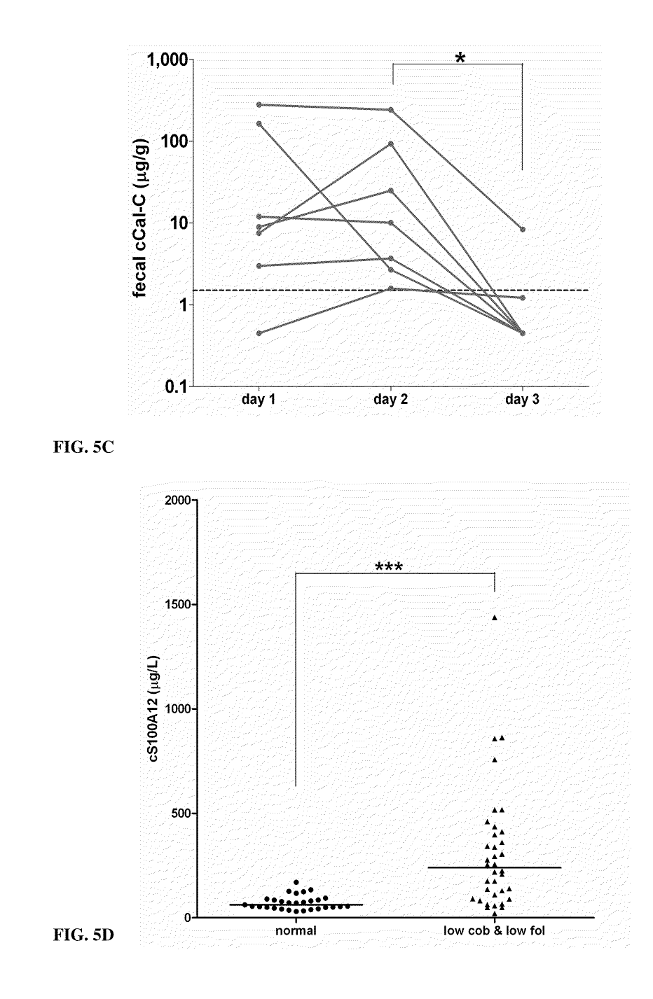

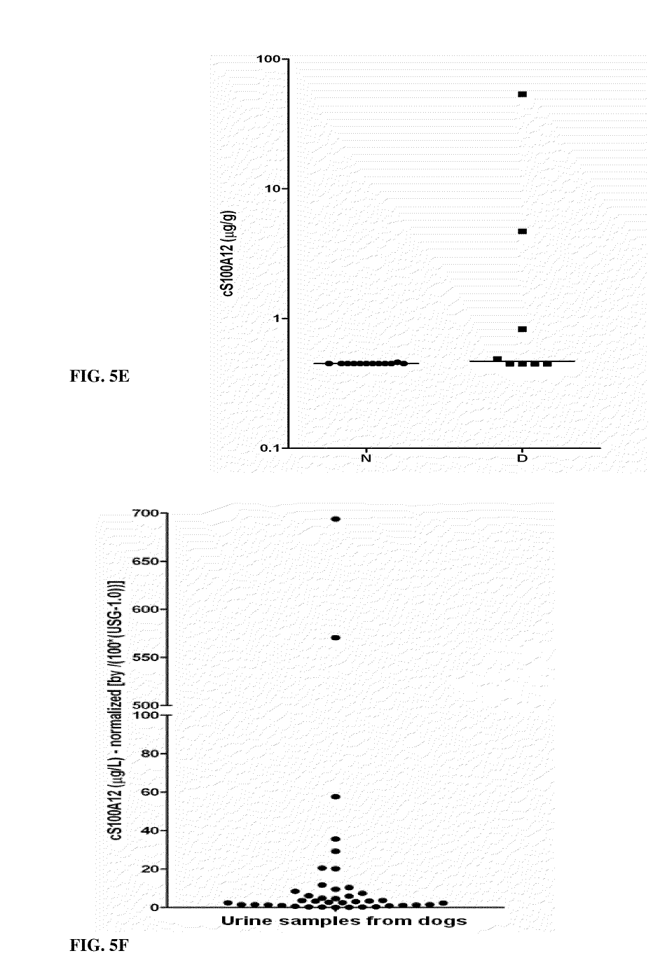

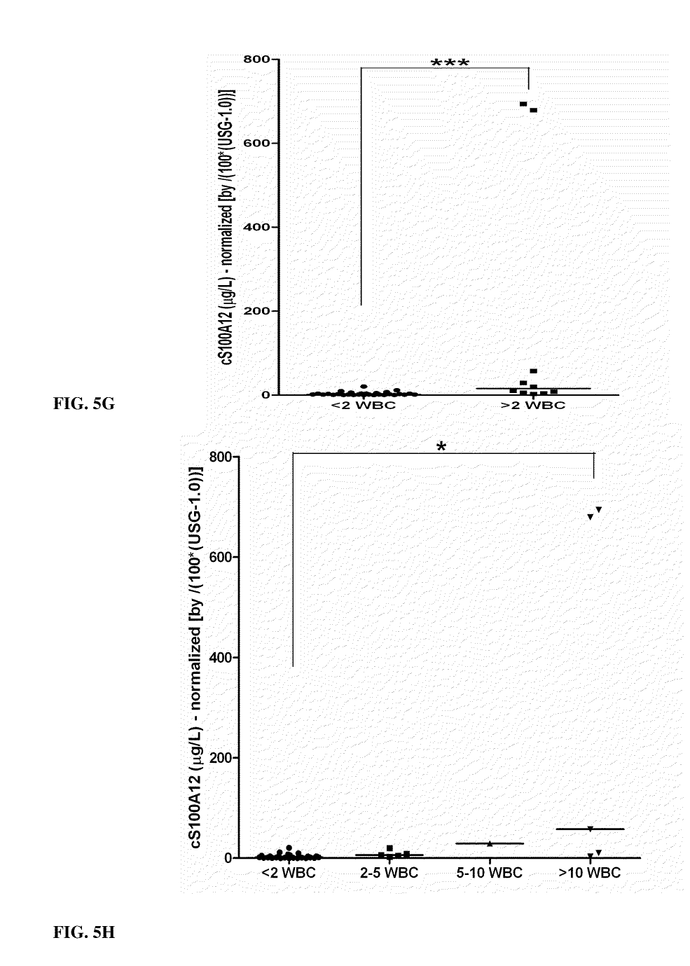

Canine S100A12 concentrations were significantly different between healthy pet dogs (median: 0.5 μg/g; range: <0.5 to 1.9 μg/g for the 3-day sample mean; median: 0.5 μg/g; range: <0.5 to 4.9 μg/g for the 3-day sample maximum) and dogs with acute hemorrhagic gastroenteritis (median: 13.2 μg/g; range: <0.5 to 279.3 μg/g; p<0.0001) (FIG. 5A and FIG. 5B). Following the initiation of therapy, canine S100A12 concentrations significantly decreased between days 2 and 3 (n=7; p=0.0162) (FIG. 5C).

Canine S100A12 concentrations were measured in serum samples from 65 dogs with severely decreased serum cobalamin (i.e., <149 ng/L) and low serum folate (i.e., <7.7 μg/L) concentrations suggesting both proximal and distal small intestinal disease, and were compared to canine S100A12 concentrations in serum from 61 healthy dogs by using a Mann-Whitney u test (statistical significance: p<0.05).

Serum canine S100A12 concentrations were significantly higher in dogs with severely decreased serum cobalamin and low serum folate concentrations (median: 208 μg/L; range: 20 to 2,291 μg/L) compared to healthy pet dogs (median: 80 μg/L; range: 30 to 300 μg/L; p<0.0001) (FIG. 5D).

Canine S100A12 concentrations were measured in fecal samples collected from eight dogs with acute diarrhea (group D), and were compared to a single fecal sample each collected from 12 healthy dogs (group N) from the same geographical area by use of a Mann-Whitney u test (statistical significance: p<0.05).

Canine S100A12 concentrations were significantly higher in dogs with acute diarrhea (median: 0.5 μg/g; range: <0.5 to 53.7 μg/g) compared to canine S100A12 concentrations in fecal samples from 12 healthy dogs (median: 13.2 μg/g; range: <0.5 to 279.3 μg/g; p<0.0001) (FIG. 5E).

Canine S100A12 concentrations were measured in urine samples from 39 dogs that were submitted for a complete urinalysis, and were normalized against urine specific gravity (USG) by use of the following equation: urinary canine S100A12 (USG normalized)={canine S100A12 in μg/L/100×(USG−1.0)) (FIG. 5F). A Mann-Whitney U test was used to compare normalized urinary canine S100A12 concentrations in urine from dogs with a white blood cell count (WBC)<2 cells/hpf (n=29) and urine samples from dogs with a WBC>2 cells/hpf (n=10) (FIG. 5G). Based on the results obtained, urine samples from dogs with >2 WBC/hpf were further divided into 3 different groups (2-5 WBC/hpf, 5-10 WBC/hpf, and >10 WBC/hpf) and were compared to canine S100A12 concentrations in urine samples with <2 WBC/hpf (FIG. 5H) by use of a Kruskal-Wallis test with a Dunn's multiple comparison test (statistical significance: p<0.05).

Canine S100A12 concentrations were significantly higher in urine with a white blood cell count (WBC)>2 cells/hpf (median: 15.4 μg/L; range: 3.1 to 693.9 μg/L) compared to urine with a WBC<2 cells/hpf (median: 1.6; range: 0.1 to 20.6; p=0.0001) (FIG. 5G). When urinary canine S100A12 concentrations were compared between urine samples with <2, 2-5, 5-10, and >10 WBC/hpf, only the difference between samples with <2 WBC/hpf and samples with >10 WBC/hpf was statistically significant (p=0.0025) (FIG. 5H).

Canine S100A12 concentrations were measured in samples of cerebrospinal fluid (CSF) from 25 dogs submitted for diagnostic purposes.

Canine S100A12 concentrations were measured in a concentration above the detection limit of the assay (i.e., >0.5 ptg/L) in five of the 25 (20%) CSF samples from dogs (FIG. 5I). The canine S100A12 concentrations in those samples were 5.4, 11.6, 21.3, 16.7, and 181.9 μg/L. The fact that canine S100A12 can be quantified in CSF suggests its potential usefulness for the diagnosis and monitoring of inflammatory diseases of the central nervous system.

FIGS. 5A-5I are graphs of canine S100A12 (calgranulin C, EN-RAGE, MRP6, p6) and uses thereof in dogs. FIGS. 5A, 5B and 5C show canine S100A12 concentrations in fecal samples collected from dogs with acute hemorrhagic gastroenteritis. FIG. 5D is a graph demonstrating serum canine S100A12 concentrations in dogs with low serum cobalamin & low serum folate concentrations. FIG. 5E is a graph showing canine S100A12 concentrations in fecal samples collected from dogs with acute diarrhea. FIGS. 5F, 5G, and 5H are graphs of showing canine S100A12 concentrations (normalized against the urine specific gravity [USG]) in urine samples from dogs with different white blood cell counts as determined by urinalysis. FIG. 5I is a graph showing canine S100A12 concentrations in cerebrospinal fluid (CSF) collected from dogs for various clinical diagnostic indications.

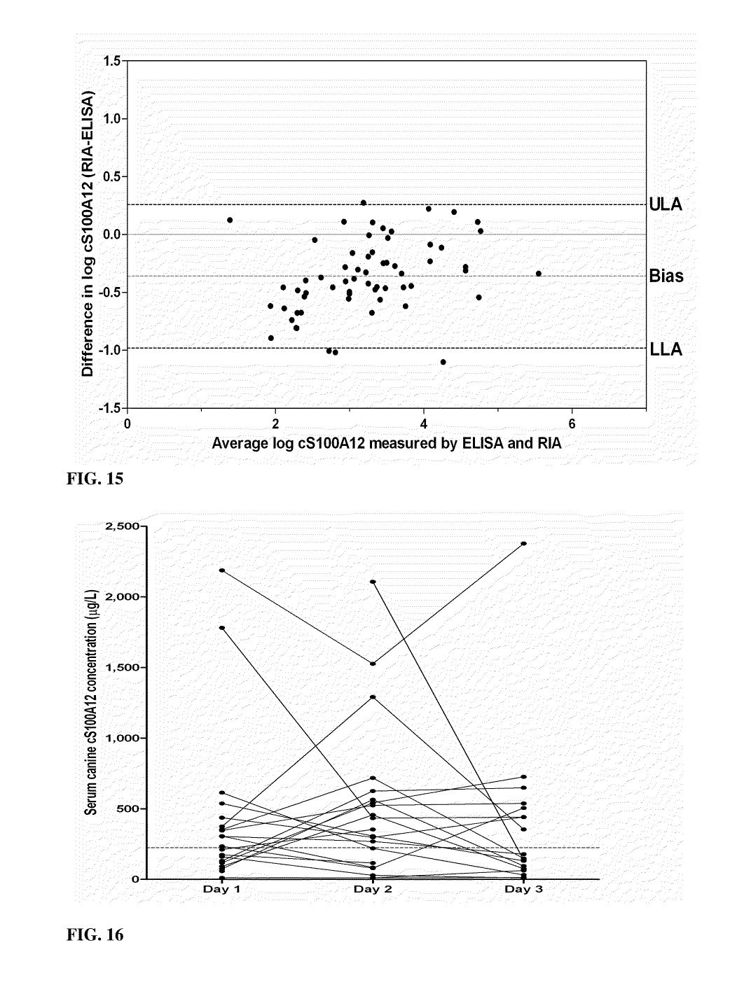

Serum canine S100A12 concentrations were measured in 22 dogs with sepsis over a 3-day period during which patients were hospitalized. Serum canine S100A12 concentrations were above the upper limit of the reference interval (225.1 μg/L) on all 3 days in 6/22 dogs (27.3%), on 2 of the 3 days in another 6/22 dogs (27.3%), and on 1 of the 3 days in 5/22 dogs (22.7%). Two of the dogs (9.1%) had no detectable serum canine S100A12 concentration (i.e., less than the 12.0 μg/L detection limit of the assay) on 2 of the 3 days, and in one of the dogs (4.5%) canine S100A12 was undetectable on 1 of the 3 days (FIG. 16).

Serum canine S100A12 concentrations were measured in dogs with chronic enteropathies (inflammatory bowel disease [IBD] or protein-losing enteropathy [PLE] or food-responsive disease [FRD]). Dogs with chronic enteropathies had significantly increased serum canine S100A12 concentrations (median: 363.2 μg/L) compared to healthy control dogs (median: 84.4 μg/L; p<0.0001) (FIG. 17A). Compared to healthy controls, serum canine S100A12 concentrations were significantly increased in dogs with IBD or PLE (median: 353.7 μg/L) or FRD (median: 364.4 μg/L; p<0.0001) but no significant difference was found between serum S100A12 concentrations between IBD or PLE and FRD dogs (p=0.9621) (FIG. 17B).

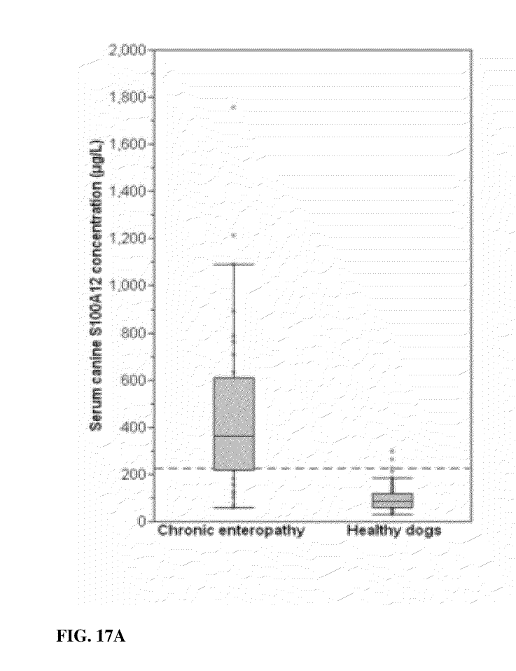

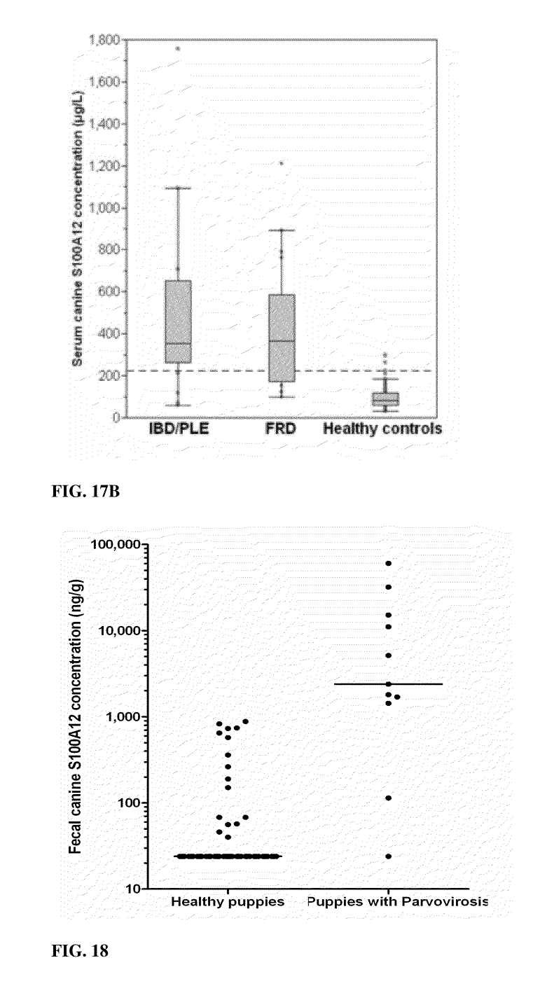

Fecal canine S100A12 concentrations were measured in puppies with parvovirosis and in healthy controls. Fecal canine S100A12 concentrations were significantly increased in puppies with parvovirosis (median: 2,393 ng/g) compared to healthy puppies (median: 24 ng/g; p<0.0001) (FIG. 18).

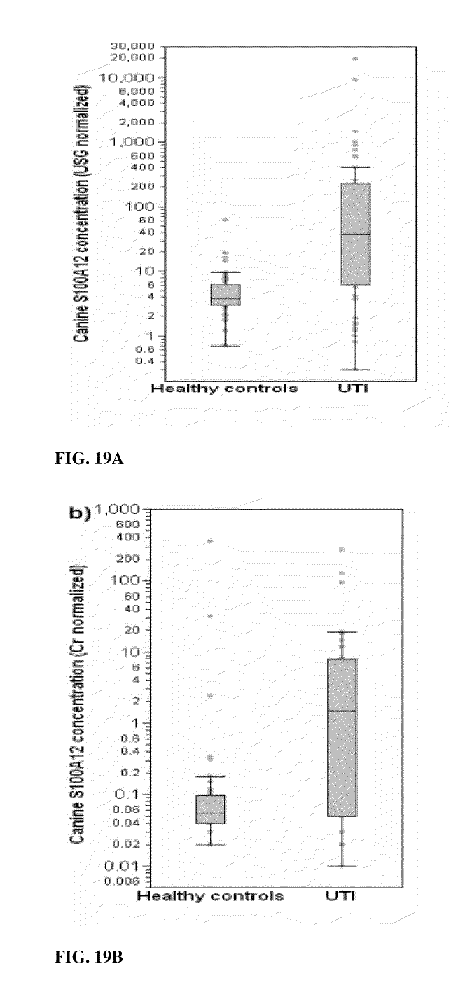

Urine canine S100A12 concentrations (normalized by urine specific gravity [USG] or urine creatinine concentration [Cr]) were measured in dogs with urinary tract infections (UTI) and healthy controls. Significantly increased USG-normalized canine S100A12 concentrations were measured in urine specimens from dogs with UTI (median: 38.3 μg/L) compared to healthy controls (median: 3.8 μg/L; p<0.0001) (FIG. 19A). Cr-normalized urine canine S100A12 concentrations were also significantly increased in these dogs with UTI (median: 1.475) compared to healthy controls (median: 0.055; p=0.0001) (FIG. 19B).

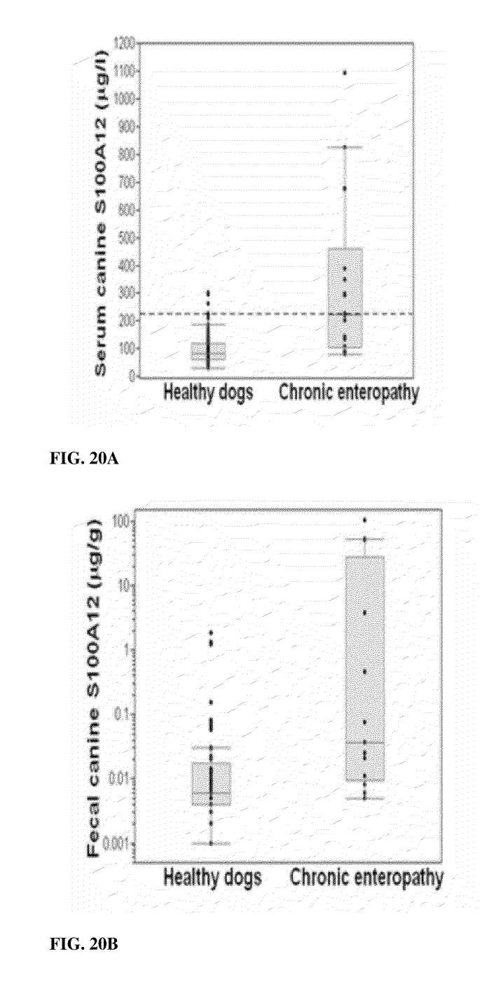

Serum and fecal canine S100A12 concentrations were measured in dogs with IBD (chronic enteropathy) compared to healthy controls. Serum canine S100A12 concentrations were significantly increased in dogs with chronic enteropathy (median: 223.0 μg/L) compared to healthy control dogs (median: 84.4 μg/L; p<0.0001) (FIG. 20A). Compared to S100A12 concentrations measured in single fecal samples (median: 6 ng/g) from 53 healthy controls, canine S100A12 concentrations were significantly increased in single fecal specimens collected from 13 dogs with chronic enteropathy (median: 36 ng/g; p=0.0015 and 0.0414, respectively) (FIG. 20B).

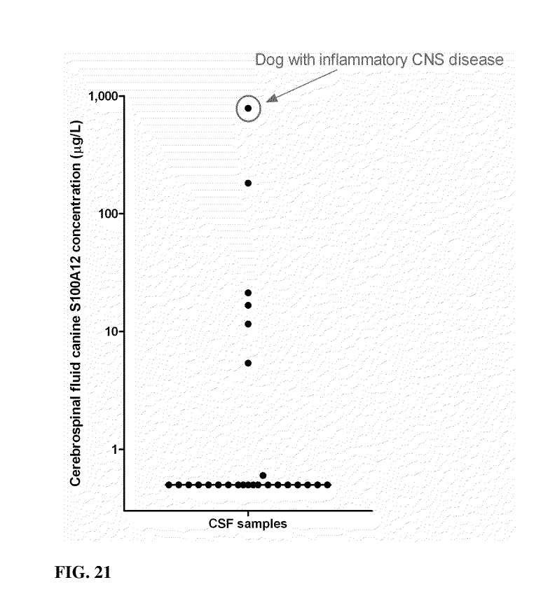

Concentrations of canine S100A12 in cerebrospinal fluid (CSF) were measured from dogs. The concentration of canine S100A12 in CSF collected from a dog with known inflammatory disease of the central nervous system (CNS) was more than four times the maximum canine S100A12 concentration measured in CSF from 25 dogs of unknown health status (FIG. 21).

It is contemplated that any embodiment discussed in this specification can be implemented with respect to any method, kit, reagent, or composition of the invention, and vice versa. Furthermore, compositions of the invention can be used to achieve methods of the invention.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

What is claimed is:

1. A method for diagnosing an inflammatory disease in an animal comprising assaying S100 calcium binding protein level in a biological sample from the animal, wherein an increased S100 calcium binding protein level is indicative of an inflammatory disease in the animal.

2. The method of claim 1, wherein the inflammatory disease is selected from the group consisting of a systemic disease, a gastrointestinal disease, a urogenital disease, and a disease of the central nervous system.

3. The method of claim 1, wherein the inflammatory disease is selected from the group consisting of inflammatory bowel disease, acute gastroenteritis, chronic gastroenteropathy, parvovirosis, urinary tract infection, inflammatory disease of the central nervous system, and sepsis.

4. The method of claim 1, wherein the biological sample is selected from a group consisting of plasma/serum, cerebrospinal fluid, urine and feces.

5. The method of claim 1, wherein the S100 calcium binding protein is S100A12.

6. The method of claim 1, wherein the animal is canine or porcine.

7. The method of claim 1, wherein the animal is a canine.

8. The method of claim 1, wherein the S100 calcium binding protein is canine S100A12.

9. The method of claim 1, wherein assaying S100 calcium binding protein level comprises a competitive immunoassay, a western blot, a radioimmunoassay (RIA), an ELISA (enzyme linked immunosorbent assay), a “sandwich” immunoassay, an immunoprecipitation assay, a precipitin reaction, a gel diffusion precipitin reaction, an immunodiffusion assay, an agglutination assay, an immunoradiometric assay, a fluorescent immunoassay, a protein A immunoassay, an immunoprecipitation assay, an immunohistochemical assay, a competition or sandwich ELISA, a radioimmunoassay, a Western blot assay, an immunohistological assay, an immunocytochemical assay, a dot blot assay, a fluorescence polarization assay, a scintillation proximity assay, a homogeneous time resolved fluorescence assay, a IAsys analysis, multiplex immunoassay, or a BIAcore analysis.

10. The method of claim 1, wherein the S100 calcium binding protein comprises 85% identity to SEQ ID NO:9 or SEQ ID NO:10.

11. An antibody that binds specifically to a canine S100A12 calcium binding protein or an immunogenic portion thereof.

12. The antibody of claim 11, wherein the antibody is a monoclonal antibody.

13. The antibody of claim 11, wherein the antibody is a polyclonal antibody.

14. A nucleic acid molecule encoding a polypeptide with at least 85% identity to SEQ ID NO:9 or 10.

15. The nucleic acid molecule of claim 14, operably linked to a heterologous promoter.

16. An expression vector comprising the nucleic acid of claim 14.

17. A cell comprising the expression vector of claim 16.

18. A kit for diagnosing an inflammatory disease in an animal comprising:
a) an antibody that specifically binds to a canine S100 calcium binding protein or an immunogenic portion thereof to form a binding complex; and
b) a detectable label.

19. The kit of claim 18, wherein the antibody comprises a monoclonal antibody or a polyclonal antibody.

20. The kit of claim 18, wherein the detectable label comprises a radioactive, enzymatic, or colorimetric label.

21. The kit of claim 18, wherein the canine S100 calcium binding protein is S100A12.

22. The kit of claim 18, wherein the kit is a competitive immunoassay, a western blot, a radioimmunoassay (RIA), an ELISA (enzyme linked immunosorbent assay), a “sandwich” immunoassay, an immunoprecipitation assay, a precipitin reaction, a gel diffusion precipitin reaction, an immunodiffusion assay, an agglutination assay, an immunoradiometric assay, a fluorescent immunoassay, a protein A immunoassay, an immunoprecipitation assay, an immunohistochemical assay, a competition or sandwich ELISA, a radioimmunoassay, a Western blot assay, an immunohistological assay, an immunocytochemical assay, a dot blot assay, a fluorescence polarization assay, a scintillation proximity assay, a homogeneous time resolved fluorescence assay, a IAsys analysis, multiplex immunoassay, or a BIAcore analysis.
